(19) United States
(12) Patent Application Publication
(10) Pub. No.: US 2025/0057589 A1
(43) Pub. Date: Feb. 20, 2025

(54) NEUROMODULATION CATHETER

(71) Applicant: Medtronic Ireland Manufacturing Unlimited Company, Dublin (IE)

(72) Inventors: Kelsey Sandquist, Cotati, CA (US); Cian Michael Ryan, Santa Rosa, CA (US); Paulina Nguyen, Santa Rosa, CA (US); Lana Woolley, Santa Rosa, CA (US); Brian Dowling, Santa Rosa, CA (US); Jaclyn N. Kawwas, Philadelphia, PA (US); Alexandra C. Dotti, Rohnert Park, CA (US); Sina Som, Santa Rosa, CA (US); Rudy A. Beasley, Rohnert Park, CA (US)

(21) Appl. No.: 18/720,963

(22) PCT Filed: Dec. 19, 2022
(86) PCT No.: PCT/EP2022/086739

§ 371 Date: Jun. 17, 2024

Related U.S. Application Data
(60) Provisional application No. 63/292,560, filed on Dec. 22, 2021.

(51) Int. Cl. A61B 18/14; A61B 18/00

(52) U.S. Cl. CPC A61B 18/1492; A61B 2018/00077; A61B 2018/00178; A61B 2018/00184; A61B 2018/00214; A61B 2018/00821; A61B 2018/1475

(57) ABSTRACT

A catheter configured to deliver energy circumferentially around an anatomical lumen in which the catheter is positioned. The catheter includes an elongate structure configured to transform between a low-profile delivery state and a radially expanded deployed state. The elongate structure mechanically supports one or more electrodes configured to emit energy and includes an outer jacket comprising a polymer. The catheter defines a material boundary adjoining the outer jacket and at least one electrode to enable a mechanical adhesion between the polymer of the outer jacket and the electrode. In examples, the polymer intrudes into one or more volumes defined by asperities in a surface of the electrode to enable the mechanical adhesion. In examples, material boundary is formed by reflowing the polymer.

### TECHNICAL FIELD

The present technology is related to neuromodulation systems, catheters and methods. In particular, various examples of the present technology are related to neuromodulation catheters having one or more electrodes.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for neuromodulation (e.g., denervation) using one or more electrodes. The present technology may be directed to, for example, renal neuromodulation, spinal neuromodulation, cardiac neuromodulation, brain neuromodulation, sacral neuromodulation, urinary neuromodulation, and/or neuromodulation techniques directed to other portions of a body. A catheter may be configured (e.g., have suitable shape and dimensions) to deliver energy (e.g., radiofrequency, pulsed field, direct electrical current) with a portion of the catheter carrying an electrode positioned in tissue or in an anatomical lumen (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, etc. and/or other anatomical lumens). The catheter may include an elongate structure (e.g., in a distal portion of the catheter) configured to transform between a low-profile delivery state and a radially expanded deployed state. The elongate structure mechanically supports one or more electrodes configured to emit energy (e.g., at least two electrodes, three electrodes, four electrodes, or the like) and includes an outer jacket comprising a polymer. The catheter defines a material boundary adjoining the outer jacket and at least one electrode to enable a mechanical adhesion between the polymer of the outer jacket and the electrode. In examples, the polymer intrudes into one or more volumes defined by asperities in a surface of the electrode to enable the mechanical adhesion. In examples, the material boundary is formed by reflowing the polymer.

In an example, a catheter comprises a neuromodulation element, which may be convertible between a low-profile delivery configuration and a radially expanded deployed configuration, the neuromodulation element comprising: an elongate structure defining a longitudinal axis and configured to have a substantially linear shape when the neuromodulation element is in the low-profile delivery configuration and configured to have a helical shape when the neuromodulation element is in the radially expanded deployed configuration, wherein the elongate structure includes an outer jacket comprising a polymer, and wherein the outer jacket defines an exterior surface of the elongate structure; and at least one electrode configured to emit energy from an active area, wherein the elongate structure mechanically supports the at least one electrode such that the exterior surface and the active area face outward from the longitudinal axis, and wherein the catheter defines a material boundary adjoining the outer jacket and the at least one electrode, wherein the material boundary enables a mechanical adhesion between the polymer and the at least one electrode.

In an example, a method for forming a neuromodulation element comprises: forming an elongate structure, wherein the elongate structure includes an outer jacket comprising a polymer, and wherein the outer jacket defines an exterior surface of the elongate structure; mechanically supporting, using the elongate structure, at least one electrode configured to emit energy from an active area such that the exterior surface and the active area face outward from the longitudinal axis, and reflowing the polymer to define a material boundary adjoining the outer jacket and the at least one electrode, wherein the material boundary enables a mechanical adhesion between the polymer and the at least one electrode. The elongate structure may define a longitudinal axis and be configured to have a substantially linear shape when the neuromodulation element is in a low-profile delivery configuration and a helical shape when the neuromodulation element is in a radially expanded deployed configuration.

In an example, a catheter is configured to deliver energy circumferentially around an anatomical lumen in which the catheter is positioned. The catheter includes an elongate structure configured to transform between a low-profile delivery state and a radially expanded deployed state. The elongate structure mechanically supports one or more electrodes configured to emit energy and includes an outer jacket comprising a polymer. The catheter defines a material boundary adjoining the outer jacket and at least one electrode to enable a mechanical adhesion between the polymer of the outer jacket and the electrode. In examples, the polymer intrudes into one or more volumes defined by asperities in a surface of the electrode to enable the mechanical adhesion. In examples, material boundary is formed by reflowing the polymer.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1 is a partially schematic illustration of a neuromodulation system configured in accordance with some examples of the present disclosure.

FIG. 2 is an exploded profile view of the catheter shown in FIG. 1.

FIG. 3 is an enlarged exploded profile view of a portion of the catheter shown in FIG. 1 taken at the location designated in FIG. 2.

FIG. 4 is a perspective view of an elongate structure of a neuromodulation element of a neuromodulation catheter configured in accordance with examples of the present disclosure.

FIG. 5 is a perspective view of an outer jacket and electrodes seated within reduced-diameter segments, in accordance with some examples of the present disclosure.

FIG. 6 is a profile view of the outer jacket shown and electrodes shown in FIG. 5 seated within reduced-diameter segments, in accordance with some examples of the present disclosure.

FIG. 7 is a profile view of the outer jacket shown in FIG. 5, in accordance with some examples of the present disclosure.

FIG. 8 is an enlarged profile view of a portion of the outer jacket shown in FIG. 5 taken at a location designated in FIG. 7.

FIG. 9 is a cross-sectional view of a portion of the outer jacket shown in FIG. 4 and an electrode positioning on the outer jacket.

FIG. 10 is a cross-sectional view of the portion of the outer jacket shown in FIG. 10 and an electrode seated within a reduced diameter segment.

FIG. 11 is a cross-sectional view illustrating material boundaries between an outer jacket, an electrode, and an electrical lead.

FIG. 12 is a cross-sectional view of an outer jacket including an internal jacket member and an external jacket member.

FIG. 13 is a cross-sectional view of the outer jacket of FIG. 12 defining one or more material boundaries.

FIG. 14 is a perspective view of an outer jacket defining one or more windows.

FIG. 15 is a perspective view of the outer jacket of FIG. 14 and one or more electrodes positioned relative to the one or more windows.

FIG. 16 is a profile view illustrating the outer jacket and electrodes of FIG. 14 defining one or more material boundaries.

FIG. 17 is a flow diagram illustrating a method of forming an elongate structure.

### DETAILED DESCRIPTION

The present technology is directed to devices, systems, and methods for neuromodulation (e.g., denervation) using one or more electrodes.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Conditions such as arrhythmias, hypertension, states of volume overload (e.g., heart failure), and progressive renal disease due to excessive activation of the renal sympathetic nervous system (SNS), may be mitigated by modulating the activity of overactive nerves (neuromodulating), for example, denervating or reducing the activity of the overactive nerves. Sympathetic nerves of the kidneys terminate in the renal blood vessels, the juxtaglomerular apparatus, and the renal tubules, among other structures. The overactive nerves may be chemically, thermally, or electrically denervated by ablating sympathetic nerve tissue in or near renal blood vessels. A chemical, thermal, or electrical energy may be delivered to the sympathetic tissue via navigating a catheter including needles and/or electrodes within the vasculature of the patient.

In neuromodulation, one or more therapeutic elements may be introduced near one or more target nerves. In renal neuromodulation, for example, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique. The plurality of therapeutic elements may be arranged around an outer perimeter of an expandable distal portion of the catheter. The neuromodulation element may be configured to contact tissue at locations where one or more therapeutic elements are configured. In an expanded or deployed configuration, if present, the neuromodulation element may be configured to contact a vessel wall or other anatomical lumen wall both proximal and distal to the locations at which a plurality of the therapeutic elements are configured. This may help approximately center the distal portion of the catheter within the vessel or other anatomical lumen, help retain the distal catheter portion in position relative to the vessel wall or other anatomical lumen wall during the neuromodulation treatment, and/or help protect the vessel wall or other anatomical lumen wall by redistributing an applied pressure between the therapeutic elements and the vessel wall.

In some examples, an ablation system includes a generator configured to generate energy and deliver energy (e.g., radiofrequency, pulsed field, direct electrical current) to tissue via one or more electrodes carried by a catheter and positioned within tissue or an anatomical lumen of a body of a patient. The energy may heat tissue to which the energy is directed (which tissue includes one or more nerves) and modulate the activity of the one or more nerves. The ablation system may be configured to deliver energy via either a monopolar or bipolar arrangement, for example. In a monopolar arrangement, a return or reference electrode may be paced on a patient's skin, and one or more of the electrodes carried by the catheter may be driven to act as active electrodes, either simultaneously or sequentially. In a bipolar arrangement, the active and return electrodes may both be carried by or attached to the catheter and introduced within the body of the patient. In some examples, a catheter includes a plurality of electrodes, and the generator and electrical connections between the generator and the electrodes can be configured for monopolar energy delivery, bipolar energy delivery, or can be controllable between monopolar energy delivery and bipolar energy delivery.

Although the present technology is herein described in many instances with reference to renal nerves and vessels, it should be understood that the present technology also has application to devices, systems and methods for neuromodulation at other anatomical sites (e.g., spinal neuromodulation, cardiac neuromodulation, brain neuromodulation, sacral neuromodulation, urinary neuromodulation, and/or neuromodulation techniques directed to other portions of a body) and their associated nerves and that such devices and systems can be configured (e.g., have suitable shape and dimensions) for such sites. For example, a catheter may be configured to deliver energy with a portion of the catheter carrying an electrode positioned in particular tissue or a particular anatomical lumen (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, etc. and/or other anatomical lumens).

In many patients, renal nerves generally follow the renal artery and branch vessels from near the aorta to a kidney. The renal nerves may be present in a wall of the renal artery and/or branch vessels and/or in tissue surrounding the renal artery and/or branch vessels. Because renal nerves may be around the renal artery and/or branch vessels and may include multiple nerves and/or nerve branches, it may be desirable to deliver energy circumferentially around the renal artery and/or branch vessels to affect as many renal nerves as possible.

In accordance with examples of the current disclosure, a catheter (e.g., an ablation catheter) is configured to deliver energy with a portion of the catheter carrying an electrode positioned in tissue or in an anatomical lumen. The catheter includes an elongate structure (e.g., in a distal portion of the catheter) including an outer jacket. The elongate structure mechanically supports one or more electrodes (e.g., at least two electrodes, three electrodes, four electrodes, or the like) and may be configured to transform between a low-profile delivery state (e.g., a substantially straight state) and a radially expanded deployed state (e.g., a spiraled or helical state). The elongate structure can be configured such in its expanded deployed state such that the electrode(s) are positioned against a wall of an anatomical lumen (e.g., a vessel). In the radially expanded deployed state of an elongate structure having multiple electrodes, the position of the electrodes along the elongate structure and the spacing between adjacent turns of the spiral or helix (e.g., a deployed electrode length) may provide a desired ablation pattern. For example, the elongate structure may be configured such that the deployed electrode length in the radially expanded deployed state is relatively small (e.g., the spiraled or helical state is relatively compressed) or relatively large (e.g., the spiraled or helical state is relatively extended). The arrangement of electrodes in the expanded deployed state may space ablations created by the electrodes from one another or the ablations may overlap. Where the ablations overlap and the deployed electrode length is sufficiently small, a substantially continuous circumferential lesion (e.g., a ring-like lesion formed by a plurality of lesions substantially overlapping in a circumferential plane) may be formed in tissue, which may reduce a likelihood of renal nerves being left untreated and improve a likelihood of success of the denervation therapy. In some examples, the elongate structure may be configured such that the deployed electrode length in the radially expanded deployed state is relatively larger (e.g., the spiraled or helical state is relatively extended), such that a substantially spiraled lesion, a plurality of focal legions, or one or more differently arranged legions may be formed in tissue.

The elongate structure defines a longitudinal axis extending from a proximal portion of the elongate structure through a distal end of the elongate structure. In examples, the longitudinal axis extends through a lumen defined by the elongate structure. The longitudinal axis may be substantially straight (e.g., when the elongate structure is in a low-profile delivery state) or may exhibit a curvature (e.g., when the elongate structure is in a radially expanded deployed state). In examples, the outer jacket defines an exterior surface defining an outermost surface of the elongate structure (e.g., outermost in a radial direction from the longitudinal axis). Each of the one or more electrodes defines an active area configured to emit the energy. The elongate structure mechanically supports the one or more electrodes such that the active area of an electrode and the exterior surface face outward from the longitudinal axis.

For example, the outer jacket may define a substantially tubular structure with the exterior surface being some portion of an outer facing surface of the tubular structure. The electrode may be a band electrode (e.g., ring shaped) with the active area being some portion of an outer facing surface of the band electrode. The electrode may substantially surround the outer jacket, such that the elongate structure mechanically supports the electrode with the active area of the electrode and the exterior surface of the outer jacket facing outward from the longitudinal axis. In some examples, the electrode is positioned with an inner lumen of the outer jacket (“jacket inner lumen”) and the elongate structure mechanically supports the electrode such that the active area of the electrode faces outward through a window defined by the outer jacket. The electrode and the outer jacket may have other shapes in other examples.

The catheter defines a material boundary adjoining the outer jacket and at least one electrode to, for example, assist in holding the electrode more securely in place when the elongate structure mechanically supports the electrode. The material boundary may enable a mechanical adhesion between at least a polymer comprising the outer jacket and a surface of the electrode (e.g., a surface other than that defining the active area of the electrode). In examples, the material boundary is defined by an edge surface of the outer jacket (“jacket edge surface”) and an edge surface of the electrode (“electrode edge surface”). The jacket edge surface may be in contact with the electrode edge surface. In examples, the jacket edge surface intrudes into one or more volumes defined by asperities of the electrode edge surface (e.g., due to a surface roughness of the electrode edge surface) to enable the mechanical adhesion. For example, the material boundary may be formed by reflowing a polymer comprising the outer jacket such that the polymer intrudes into the one or more volumes.

For example, in some examples, the outer jacket may define one or more longitudinally spaced reduced diameter segments (e.g., recesses) in the exterior surface of the outer jacket. An electrode may be seated within a reduced-diameter segment to cause the active area of an electrode and the exterior surface to face outward from the longitudinal axis. The catheter may define the material boundary between an jacket edge surface defining the recess and an electrode edge surface when the electrode is seated within the recess. In some examples, the outer jacket defines one or more windows defining a passage from a jacket inner lumen to the exterior surface of the outer jacket. An electrode may be positioned at least partially within the jacket inner lumen such that the active area of the electrode faces outward through the window. The catheter may define the material boundary between an jacket edge surface defining the window and an electrode edge surface when the electrode is positioned at least partially within the jacket inner lumen.

The catheter may include one or more electrical leads electrically connected to the one or more electrodes. The catheter may be configured such that each individual electrical lead is electrically connected to at least one electrode. In examples, the individual electrical lead is electrically connected to a single electrode. The catheter may define one or more material boundaries adjoining the electrical lead (e.g., an insulative cover of the electrical lead) and the outer jacket to assist in holding the electrical lead more securely in place to, for example, assist in maintaining the electrical connection to the electrode. The one or more material boundaries adjoining the electrical lead and the outer jacket may be formed by reflowing the polymer comprising the outer jacket such that the polymer contacts at least some portion of an outer surface of the electrical lead (e.g., an outer surface of the insulative cover). In some examples, an edge of the outer jacket may intrude into one or more volumes defined by asperities of the electrical lead (e.g., the insulative cover) to assist the mechanical adhesion of the material boundary adjoining the electrical lead and the outer jacket.

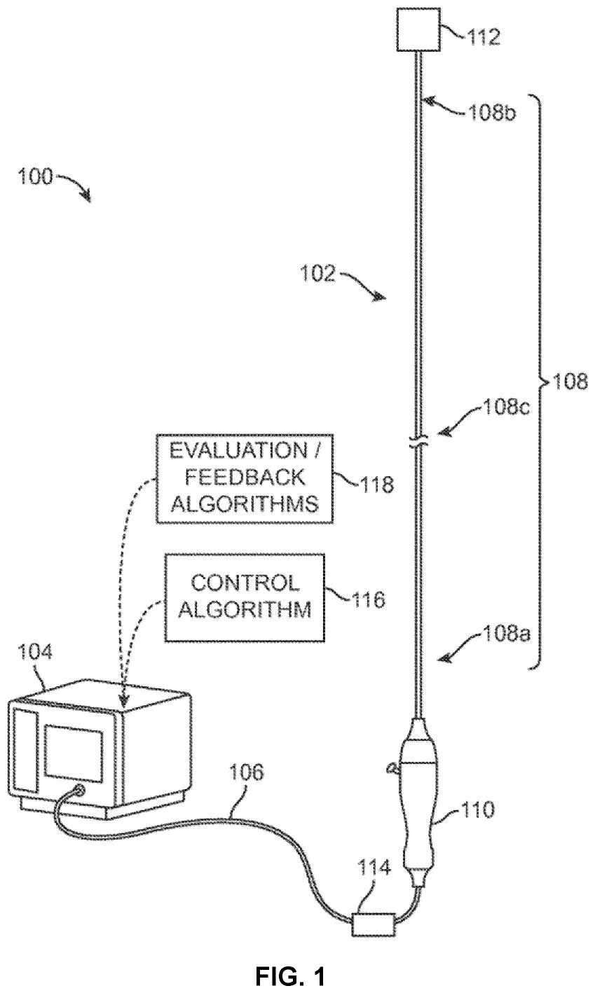

FIG. 1 is a partially schematic perspective view illustrating a medical system 100 configured in accordance with some examples of the present disclosure. Medical system 100 includes a neuromodulation catheter 102, a generator 104, and a cable 106 extending between catheter 102 and generator 104. Neuromodulation catheter 102 includes an elongate shaft (also referred to as an elongate body) 108 having a proximal portion 108a, a distal portion 108b, and an optional intermediate portion 108c between proximal portion 108a and distal portion 108b. Neuromodulation catheter 102 may further include a handle 110 operably connected to shaft 108 via proximal portion 108a and a neuromodulation element 112 (shown schematically in FIG. 1that is part of or attached to distal portion 108b. Shaft 108 is configured to locate the neuromodulation element 112 at a treatment location within or otherwise proximate to an anatomical lumen (e.g., a blood vessel, a duct, an airway, or another naturally occurring anatomical lumen within the human body). In some examples, shaft 108 is configured to locate neuromodulation element 112 at an intraluminal (e.g., intravascular) location. Neuromodulation element 112 may be configured to provide or support a neuromodulation treatment at the treatment location. Shaft 108 and neuromodulation element 112 may measure 2, 3, 4, 5, 6, or 7 French or other suitable sizes.

Intraluminal delivery of neuromodulation catheter 102 may include percutaneously inserting a guidewire (not shown) into an anatomical lumen of a patient and moving shaft 108 and neuromodulation element 112 along the guide wire until neuromodulation element 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

Generator 104 is configured to control, monitor, supply, and/or otherwise support operation of neuromodulation catheter 102. In other examples, neuromodulation catheter 102 may be self-contained or otherwise configured for operation independent of generator 104. When present, generator 104 is configured to generate a selected form and/or magnitude of energy for delivery to tissue at a treatment location via neuromodulation element 112. For example, generator 104 can be configured to generate energy (e.g., monopolar and/or bipolar energy). In other examples, generator 104 may be another type of device configured to generate and deliver another suitable type of energy to neuromodulation element 112 for delivery to tissue at a treatment location via electrodes (not shown) of neuromodulation element 112.

Along cable 106 or at another suitable location within medical system 100, medical system 100 may include a control device 114 configured to initiate, terminate, and/or adjust operation of one or more components of neuromodulation catheter 102 directly and/or via generator 104. Generator 104 may be configured to execute an automated control algorithm 116 and/or to receive control instructions from an operator. Similarly, in some implementations, generator 104 is configured to provide feedback to an operator before, during, and/or after a treatment procedure via an evaluation/feedback algorithm 118.

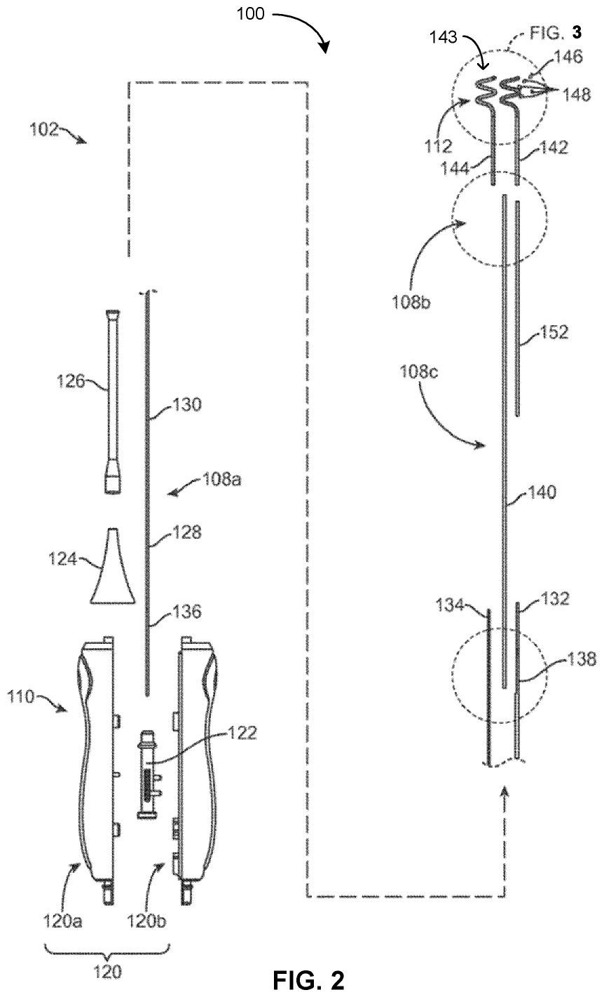

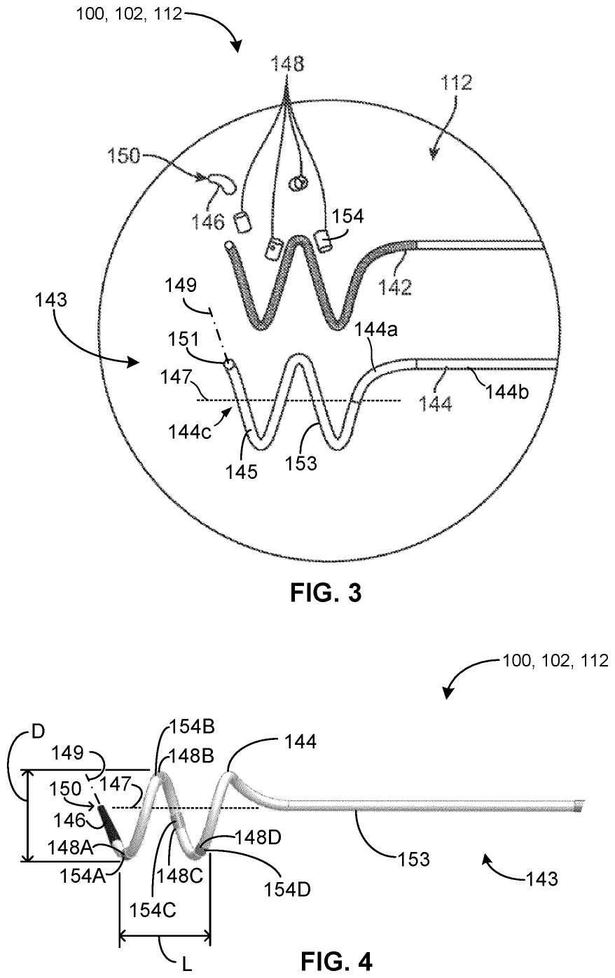

FIG. 2 is an exploded profile view of the catheter 102 of FIG. 1. FIG. 3 is an enlarged exploded profile view of portions of the catheter 102 taken at the location designated in FIG. 2. With reference to FIG. 2, handle 110 can include mating shell segments 120 (individually identified as shell segments 120a, 120b) and a connector 122 (e.g., a luer connector) operably positioned between the mating shell segments 120. Handle 110 can further include a distally tapered strain-relief element 124 operably connected to distal ends of shell segments 120. Catheter 102 can include a loading tool 126 configured to facilitate loading the catheter 102 onto a guidewire (not shown). When assembled, shaft 108 can extend through coaxial lumens (also not shown) of strain-relief element 124 and loading tool 126, respectively, and between shell segments 120 to the connector 122.

Shaft 108 can be constructed in a variety of ways. Although various options for constructing shaft 108 are provided herein, they are only examples and others are also possible. As an example, shaft 108 can include an assembly of parallel (e.g., concentric, coaxial, and/or adjacent) tubular segments. Shaft 108 can have a plurality of segments, e.g., a proximal portion 108a, a distal portion 108b, and/or an intermediate portion 108c. Shaft 108 can be constructed for use with a guidewire in over-the-wire and rapid exchange implementations. Shaft 108 can include, for example, a proximal hypotube segment 128, a proximal jacket 130, an electrically insulative tube 132, and a guidewire tube 134. In some examples, electrically insulative tube 132 and guidewire tube 134 can be disposed side-by-side within proximal hypotube segment 128. In some examples, guidewire tube 134 may be disposed alongside proximal hypotube segment 128. In some examples, guidewire tube 134 may be disposed at least partially within proximal hypotube segment 128. Electrically insulative tube 132 can be configured to carry electrical leads (not shown) and to electrically insulate the electrical leads from proximal hypotube segment 128 or other components. Guide-wire tube 134 can be configured to carry a guide wire (not shown). Proximal jacket 130 can be disposed around some or all of an outer surface of proximal hypotube segment 128. Proximal hypotube segment 128 can include a proximal stem 136 at its proximal end and a distal skive 138 at its distal end. Electrically insulative tube 132 and guide-wire tube 134 can extend distally beyond the distal skive 138. Shaft 108 can include an intermediate tube 140 beginning proximally at a region of the shaft 108 at which electrically insulative tube 132 distally emerges from proximal hypotube segment 128. Intermediate tube 140 can be more flexible than proximal hypotube segment 128. At the region of shaft 108 at which electrically insulative tube 132 distally emerges from proximal hypotube segment 128, intermediate tube 140 can be coaxially aligned with proximal hypotube segment 128 so as to receive the electrically insulative tube 132. Intermediate tube 140 can extend distally to distal end portion 108b of shaft 108. Electrically insulative tube 132 can distally terminate within intermediate tube 140. Guide-wire tube 134 can extend through the entire length of intermediate tube 140. At a distal end of intermediate tube 140, shaft 108 can be operably connected to the neuromodulation element 112.

Neuromodulation element 112 includes an elongate structure 143 including an outer jacket 144 and electrodes 148. In FIGS. 2 and 3, the neuromodulation element 112 is shown in a radially expanded deployed state. The neuromodulation element 112 can be movable from a low-profile delivery state to the radially expanded deployed state. In examples, in the radially expanded deployed state, neuromodulation element 112 (e.g., elongate structure 143) defines a shape in which a transition region 144a (FIG. 3) of neuromodulation element 112 transitions from a straight portion 144b of neuromodulation element 112 to a helical portion 144c of neuromodulation element 112. In the radially expanded deployed state, neuromodulation element 112 (e.g., helical portion 144c) may substantially define a helical form curved around a central axis 147. In examples, neuromodulation element 112 (e.g., helical portion 144c), in the radially expanded deployed state, defines central axis 147.

Elongate structure 143 defines a longitudinal axis 149 extending from a proximal portion of elongate structure 143 (e.g., straight portion 144b) through a distal end 151 of elongate structure 143. In examples, outer jacket 144 defines distal end 151. In some examples, longitudinal axis 149 extends through an inner lumen 145 defined by outer jacket 144 (“jacket inner lumen 145”). Hence, longitudinal axis 149 may be substantially straight (e.g., when elongate structure 143 is in a low-profile delivery state) or may exhibit a curvature (e.g., when elongate structure 143 is in a radially expanded deployed state). In examples, outer jacket 144 defines an exterior surface 153 (“jacket exterior surface 153”) defining an outermost surface of elongate structure 143 (e.g., outermost in a radial direction from longitudinal axis 149). Each of electrodes 148 may define an active area configured to emit energy. Elongate structure 143 is configured to mechanically support electrodes 148 such that the active area of an electrode and jacket exterior surface 153 face outward from longitudinal axis 149. Electrodes 148 may be formed from any suitable electrically conductive material. The electrically conductive material may be biocompatible. For example, electrodes 148 may include gold, platinum/iridium, or the like.

In some examples, neuromodulation catheter 102 includes a shape memory structure 142 configured to urge elongate structure 143 into the radially expanded deployed state. Shape memory structure 142 may be, for example, a hypotube (e.g., a cut hypotube), a Nitinol-based structure, such as a helical hollow strand (HHS®) structure available from Fort Wayne Metals, Fort Wayne, Indiana, or another shape memory material having a shape that is more helical (spiral) than its shape when the neuromodulation element 112 is in the low-profile delivery state. In examples, outer jacket 144 is substantially disposed around at least a portion of an outer surface of shape memory structure 142. In some examples, shape memory structure 142 extends through jacket inner lumen 145. In examples, one or more electrical leads electrically coupled to electrodes 148 may respectively extend through the outer jacket 144 (e.g., between at least an interior surface of outer jacket 144 and an outer surface of shape memory structure 142). A distal portion or end of guidewire tube 134 may connect to a proximal portion or end of shape memory structure 142, and/or may extend within a lumen defined by shape memory structure 142.

In some examples, neuromodulation element 112 and/or shape memory structure 142 has the more helical shape when at rest and is configured to be urged into the less helical shape by an external sheath (not shown) or an internal guidewire (not shown). For example, neuromodulation element 112 and/or shape memory structure 142 may be urged to the less helical shape (e.g., the low-profile delivery state) by introducing the guidewire through guidewire tube 134 and an lumen defined by neuromodulation element 112 or shape memory structure 142. Neuromodulation catheter 102 may be advanced through an anatomical lumen (e.g., vessels of a patient) to position distal portion 108b and neuromodulation element 112 at a treatment site. The guidewire then may be retracted proximally from at least distal portion 108b and neuromodulation element 112 to allow neuromodulation element 112 and/or shape memory structure 142 to recover toward or to the more helical shape and transition neuromodulation element 112 to a more helical shape.

In some examples, neuromodulation element 112 and/or shape memory structure 142 may be formed to define a shape in which transition region 142a is shaped to maintain tangency between straight portion 142b and helical portion 142c. In other words, as seen in the side view of FIG. 3, straight portion 142b may sit at approximately the same radial distance from central axis 147 as the coils of helical portion 142c. Alternatively, or additionally, transition region 142a may include a curve that transitions from straight portion 142b to helical portion 142c gradually and along an arc of a circle traced by helical portion 142c when viewing an end view of shape memory structure 142. In this way, neuromodulation element 112 and/or shape memory structure 142 may omit any transverse sections (e.g., sections that are transverse to central axis 174 of helical portion 142c). This may facilitate advancing the guidewire through the lumen of shape memory structure 142 when shape memory structure is in the more helical shape (e.g., in the radially expanded deployed state).

Neuromodulation element 112 may include a second electrically insulative tube 152 disposed around an outer surface of shape memory structure 142 to, for example, electrically separate the electrodes 148 from shape memory structure 142. Shape memory material 142 may be configured to urge second electrically insulative tube 152 into a shape conforming to the radially expanded deployed state and/or the low-profile delivery state of neuromodulation element 112 when second electrically insulative tube 152 is disposed around shape memory structure 142. In some examples, second electrically insulative tube 152 extends at least partially through jacket inner lumen 145. In some examples, the one or more electrical leads electrically coupled to electrodes 148 may respectively extend between outer jacket 144 and second electrically insulative tube 152 (e.g., when second electrically insulative tube 152 extends at least partially through jacket inner lumen 145). First electrically insulative tube 132 and/or second electrically insulative tube 152 may be made at least partially (e.g., predominantly or entirely) of polyimide and polyether block amide, respectively. In other examples, first electrically insulative tube 132 and/or second electrically insulative tube 152 are made of other suitable (e.g., insulative) materials. A distal portion or end of guidewire tube 134 may connect to a proximal portion or end of shape memory structure 142, and/or may extend within a lumen defined by shape memory structure 142.

In some examples, instead of or in addition to using the combination of a guidewire and shape memory structure 142 to transition distal portion 108b from the low-profile delivery state to the radially expanded deployed state, other actuating mechanisms may be used. For example, a pull wire may be attached near the distal tip of distal portion 108b and axial forces may be used to transition distal portion 108b from the low-profile delivery state to the radially expanded deployed state (e.g., a proximally directed axial force on the pull wire may transition distal portion 108b from the low-profile delivery state to the radially expanded deployed state, and a relaxation of the proximally directed axial force on the pull wire may transition distal portion 108b from the radially expanded deployed state to the low-profile delivery state). As another example, a push member may be attached near the distal tip of distal portion 108b and axial forces may be used to transition distal portion 108b from the low-profile delivery state to the radially expanded deployed state (e.g., a proximally directed axial force on the push member may transition distal portion 108b from the low-profile delivery state to the radially expanded deployed state, and a distally directed axial force on the push member may transition distal portion 108b from the radially expanded deployed state to the low-profile delivery state).

FIG. 4 is a perspective view of neuromodulation element 112 in a radially expanded deployed state with elongate structure 143 mechanically supporting electrodes 148. Electrodes 148 include a first electrode 148A, a second electrode 148B, a third electrode 148C, and a fourth electrode 148D (collectively, “electrodes 148”), with the electrodes defining a first active area 154A, a second active area 154B, a third active area 154C, and a fourth active area 154D, respectively (collectively, “active areas 154”). Active areas 154 are configured to emit energy (e.g., to a wall of an anatomical lumen). Elongate structure 143 mechanically supports electrodes 148 such that jacket exterior surface 153 and active area 154A, active area 154B, active area 154C, and/or active area 154D face outward from longitudinal axis 149. Electrodes 148 may be configured to deliver energy (e.g., some portion of the energy generated by generator 104). In examples, electrodes 148 are be configured to deliver (e.g., emit) the energy using active area 154A, 154B, 154C, 154D.

Neuromodulation element 112 may include any suitable number of electrodes 148. For example, neuromodulation element 112 may include at least two electrodes, at least three electrodes, at least four electrodes, exactly three electrodes, exactly four electrodes, or the like. The number of electrodes may be selected based on one or more of a variety of factors, including, for example, a number of channels provided by generator 104 (FIG. 1), desired flexibility of elongate structure 143, desired continuity (e.g., circumferential continuity) or shape of the energy field delivered by electrodes 148, or the like. Further, although some examples and techniques discussed herein may refer to a single electrode (e.g., electrode 148A, electrode 148B, electrode 148D, or electrode 148D) and/or a single active area (e.g., active area 154A, active area 154B, active area 154C, or active area 154C), the examples and techniques may apply to any of electrode 148A, electrode 148B, electrode 148C, electrode 148D or another electrode, and/or any combination thereof, and may apply to any of active area 154A, active area 154B, active area 154C, active area 154D or another active area, and/or any combinations thereof. In examples, neuromodulation element 112 may include a distally tapering atraumatic tip 146, which may include a distal opening 150 configured to allow a guidewire (not shown) to pass through the opening 150.

Electrodes 148 may be spaced along outer jacket 144 with any desired spacing (e.g., first spacing S1 and/or second spacing S2 (FIG. 7)). The spacing between adjacent electrodes 148 may be measured from a point on one electrode (e.g., a proximal end, a distal end, or a longitudinal center of electrode) to the same point on an adjacent electrode (e.g., from a proximal end, a distal end, or a longitudinal center of the adjacent electrode). The spacing between adjacent electrodes 148 may affect a positioning of electrodes 148 circumferentially about the helical or spiral shape in the radially expanded deployed configuration (and, thus, the wall of the anatomical lumen in which neuromodulation element 112 is deployed). As such, the spacing between adjacent electrodes 148 may be selected based on a deployed diameter (e.g., diameter D) of neuromodulation element 112 or a range of deployed diameters of neuromodulation element 112. For example, the spacing between adjacent electrodes 148 may be selected to achieve substantially equal distribution of electrodes 148 about a circumference of the anatomical lumen (e.g., vessel) in which neuromodulation element 112 is deployed. In examples, the deployed diameter D is between about 3 mm and about 8 mm.

In the radially expanded deployed state, elongate structure 143 may be configured to define a deployed electrode length L. In examples, the deployed electrode length L may be a distance, measured along longitudinal axis 147, when elongate structure 143 is in the radially expanded deployed state. In examples, the deployed electrode length L may be a distance (e.g., a spacing) between adjacent turns of a spiral or helix described and/or exhibited by elongate structure 143 when elongate structure 143 is in the radially expanded deployed state. In some examples, the deployed electrode length L may be a distance between a proximal-most point of the proximal-most electrode used to deliver neuromodulation energy (e.g., fourth electrode 148D) and a distal-most point of the distal-most electrode used to deliver neuromodulation energy (e.g., first electrode 148A).

Elongate structure 143 may be configured to define any deployed electrode length L. In examples, elongate structure 143 may be configured to such that the deployed electrode L length allows for one or more ablation patterns that may be desired by a clinician. For example, elongate structure 143 may be configured such that deployed electrode length L is relatively small (e.g., the spiraled or helical state is relatively compressed). By delivering energy in such a manner, a substantially continuous circumferential lesion (e.g., a ring-like lesion formed by a plurality of lesions substantially overlapping in a circumferential plane) may be formed in tissue, which may reduce a likelihood of renal nerves being left untreated and improve a likelihood of success of the denervation therapy. In some examples, elongate structure 143 may be configured such that the deployed electrode length L is relatively larger (e.g., the spiraled or helical state is relatively extended), such that a substantially spiraled lesion, a plurality of focal legions, or one or more differently arranged legions may be formed in tissue.

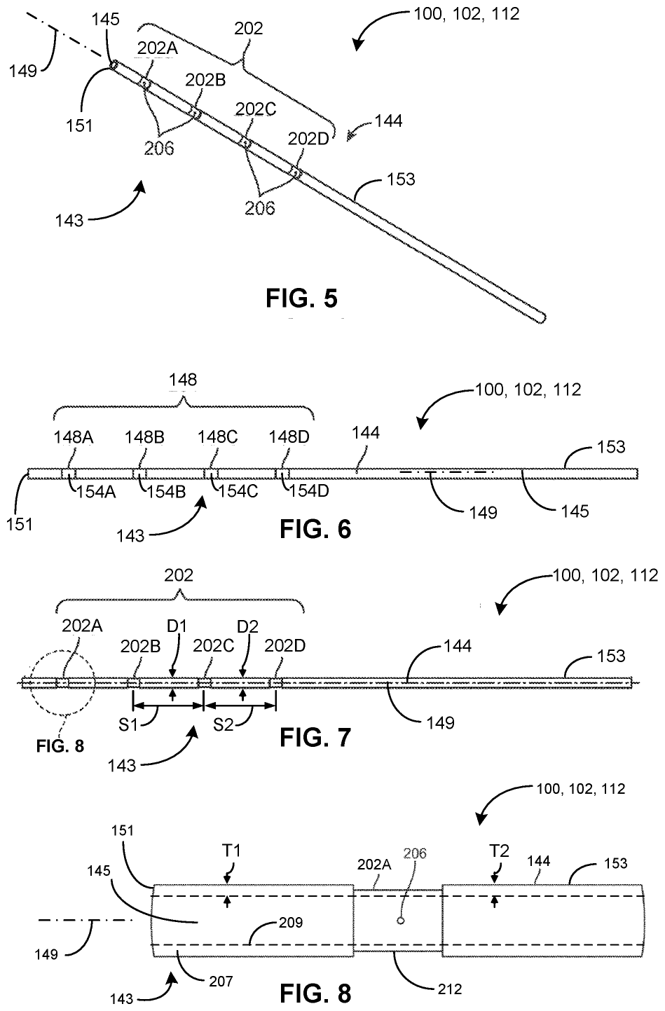

FIG. 5 is a perspective view of outer jacket 144 of a neuromodulation element configured in accordance with some examples of the present disclosure. In some examples, outer jacket 144 may include reduced-diameter segments 202 (individually identified as reduced-diameter segments 202a-202d) extending into and/or defined by its jacket exterior surface 153. FIG. 6 is a profile view of outer jacket 144 and electrode 148A, electrode 148B, electrode 148C, and electrode 148D respectively seated in the reduced-diameter segments 202. Electrodes 148 may be band electrodes. FIG. 7 is a profile view of the outer jacket 144 without electrodes 148. FIG. 8 is an enlarged profile view of a portion of the outer jacket 144 taken at a location designated in FIG. 7.

With reference to FIGS. 5-8 together, outer jacket 144 may be substantially tubular (e.g., tubular or nearly tubular to the extent permitted by manufacturing tolerances) and configured to be disposed around at least a portion of an outer surface of shape memory structure 142 (FIGS. 2 and 3). Reduced-diameter segments 202 may be insets, pockets, grooves, or other suitable structural features configured to respectively position or seat electrodes 148. In examples, a reduced-diameter segment is defined (e.g., bounded by) an edge surface defined by of outer jacket 144, such as edge surface 205 of outer jacket 144 (“jacket edge surface 205”) defining reduced-diameter segment 202a. Jacket edge surface 205 may be contiguous with and/or a portion of jacket exterior surface 153. In some examples, reduced-diameter segments 202 and/or one or more jacket edge surfaces extend around an entire circumference of outer jacket 144. In the illustrated example, outer jacket 144 includes four reduced-diameter segments 202 spaced apart along longitudinal axis 149. Alternatively, the outer jacket 144 can include one, two, three, five, six or a greater number of reduced-diameter segments 202. Reduced-diameter segments 202 may be spaced apart at equal distances or at different distances.

Outer jacket 144 may include a body 207 (“jacket body 207”) defining jacket exterior surface 153. In examples, jacket body 207 defines reduced-diameter segments 202 and/or jacket edge surface 205. In examples, outer jacket 144 (e.g., jacket body 207”) defines an interior surface 209 (“jacket interior surface 209”) opposite jacket exterior surface 153 defining jacket inner lumen 145. Jacket interior surface 209 is illustrated in dashed lines in FIG. 7. Outer jacket 144 may include a plurality of slots defining a passage from jacket inner lumen 145 to a reduced-diameter segment 202, such as slot 206 defining a passage from jacket inner lumen 145 to reduced-diameter segment 202a. In examples, slot 206 defines a passage from jacket interior surface 209 to jacket edge surface 205. Slot 206 may be configured to provide passage for an electrical lead extending through jacket inner lumen 145 to an electrode (e.g., electrode 148A) seated in reduced-diameter segment 202a. In examples, neuromodulation element 112 includes the electrical lead.

Outer jacket 144 may define any spacing between adjacent reduced diameter segments 202. For example, outer jacket 144 may define a first spacing S1 between reduced diameter segment 202B and reduced diameter segment 202C and a second spacing S2 between reduced diameter segment 202C and reduced diameter segment 202D. In some examples, outer jacket 144 may define spacings between adjacent reduced diameter segments 202 to be substantially equal over a displacement defined by (e.g., parallel to) longitudinal axis 149, such that, for example, first spacing S1 is substantially equal to second spacing S2. In some examples, outer jacket 144 may define spacings between adjacent reduced diameter segments 202 to vary between adjacent reduced diameter segments. For example, first spacing S1 may be greater than or less than second spacing S2. In some examples, outer jacket 144 defines varied spacings between adjacent reduced diameter segments to cause active areas 154 to face outward from central axis 147 (FIGS. 3-4) when neuromodulation element 112 is in a radially expanded deployed state.

Jacket exterior surface 153 may define any diameter between adjacent reduced diameter segments 202. For example, jacket exterior surface 153 may define a first diameter D1 between reduced diameter segment 202B and reduced diameter segment 202C and a second diameter D2 between reduced diameter segment 202C and reduced diameter segment 202D. In some examples, jacket exterior surface 153 may define diameters between adjacent reduced diameter segments 202 to be substantially equal such that, for example, first diameter D1 is substantially equal to second diameter D2. In some examples, jacket exterior surface 153 may define diameters which vary over jacket exterior surface 153. For example, first diameter D1 may be greater than or less than second diameter D2. In some examples, jacket exterior surface 153 defines varied diameters to cause active areas 154 to face outward from central axis 147 (FIGS. 3-4) when neuromodulation element 112 is in a radially expanded deployed state. In some examples, jacket exterior surface 153 defines a decreasing diameter as outer jacket 144 extends distally to, for example, cause active areas 154 to face outward from central axis 147 (FIGS. 3-4) when neuromodulation element 112 is in a radially expanded deployed state.

Outer jacket 144 may define any thickness between jacket exterior surface 153 and jacket interior surface 209. For example, outer jacket 144 may define a thickness T1 between reduced diameter segment 202A and structure distal end 151 and a second thickness T2 between reduced diameter segment 202A and reduced diameter segment 202B. In some examples, outer jacket 144 may define thickness to be substantially equal such that, for example, first thickness T1 is substantially equal to second thickness T2. In some examples, outer jacket 144 may define thicknesses which vary over a length of outer jacket 144. For example, first thickness T1 may be greater than or less than second thickness T2. In some examples, outer jacket 144 defines varied thicknesses to cause active areas 154 to face outward from central axis 147 (FIGS. 3-4) when neuromodulation element 112 is in a radially expanded deployed state. In some examples, outer jacket 144 defines a decreasing thickness as outer jacket 144 extends distally to, for example, cause active areas 154 to face outward from central axis 147 (FIGS. 3-4) when neuromodulation element 112 is in a radially expanded deployed state.

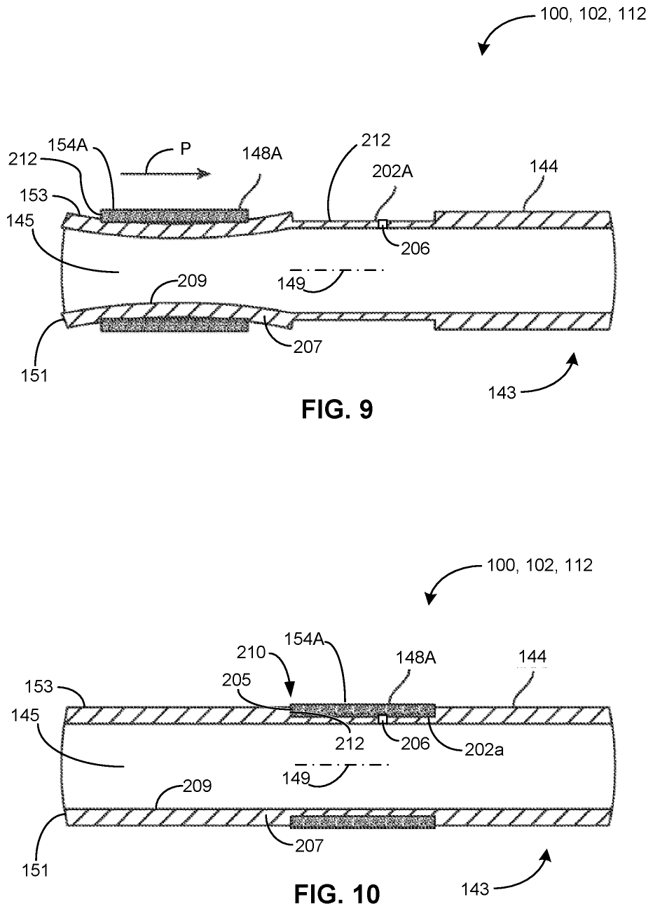

FIG. 9 illustrates outer jacket 144 resiliently deformed inwardly as electrode 148A is moved in the direction P toward reduced-diameter segment 202a to seat within reduced-diameter segment 202a. In examples, electrode 148A is a band electrode forming a closed loop circumferentially around outer jacket 144 when electrode 148A is seated within reduced diameter segment 202a. In at least some cases, a minimum inner cross-sectional dimension (e.g., an inner diameter) of electrode 148A is smaller than a maximum outer cross-sectional dimension (e.g., an outer diameter) of outer jacket 144 between the reduced-diameter segments 202. To facilitate assembly, outer jacket 144 between the reduced-diameter segments 202 can be resilient in response to peristaltic deflection of a magnitude corresponding to a difference between the maximum outer cross-sectional dimension of outer jacket 144 between the reduced-diameter segments 202 and the minimum inner cross-sectional dimension of electrodes 148a. Suitable materials for the outer jacket 144 include polymer blends including polyurethane and polysiloxane, among others. In some examples, a maximum outer cross-sectional dimension (e.g., an outer diameter) of electrode 148A and the maximum outer cross-sectional dimension of outer jacket 144 between the reduced-diameter segments 202 can be at least generally equal (e.g., within 5%, 3%, or 2% of one another).

FIG. 10 illustrates outer jacket 144 with electrode 148A seated within reduced diameter segment 202a. With electrode 148A seated within reduced diameter segment 202a, elongate structure 143 (e.g., outer jacket 144) mechanically supports electrode 148A such that jacket exterior surface 153 and active area 154 face outward from longitudinal axis 149. Catheter 102 defines a material boundary 210 adjoining outer jacket 144 and electrode 148A to, for example, assist in holding electrode 148A more securely in place when elongate structure 143 mechanically supports electrode 148A. Material boundary 210 may enable a mechanical adhesion between outer jacket 144 and electrode 148A. In examples, material boundary 210 is defined by jacket edge surface 205 and an edge surface 212 of electrode 148A (“electrode edge surface 212”), Jacket edge surface 205 may be in contact with electrode edge surface 212 when elongate structure 143 mechanically supports electrode 148A (e.g., when electrode 148A is seated within reduced diameter segment 202a). In examples, jacket edge surface 205 defines reduced diameter segment 202a. Jacket edge surface 205 may include at least some portion of and/or be contiguous with jacket exterior surface 200. In examples, electrode edge surface 212 is at least a portion of a surface of electrode 148A not including active area 154. In examples, jacket edge surface 205 intrudes into one or more volumes defined by asperities of electrode edge surface 212 to enable the mechanical adhesion. For example, the material boundary 210 may be formed by reflowing a polymer comprising outer jacket 144 such that jacket edge surface 205 intrudes into the one or more volumes defined by electrode 148A.

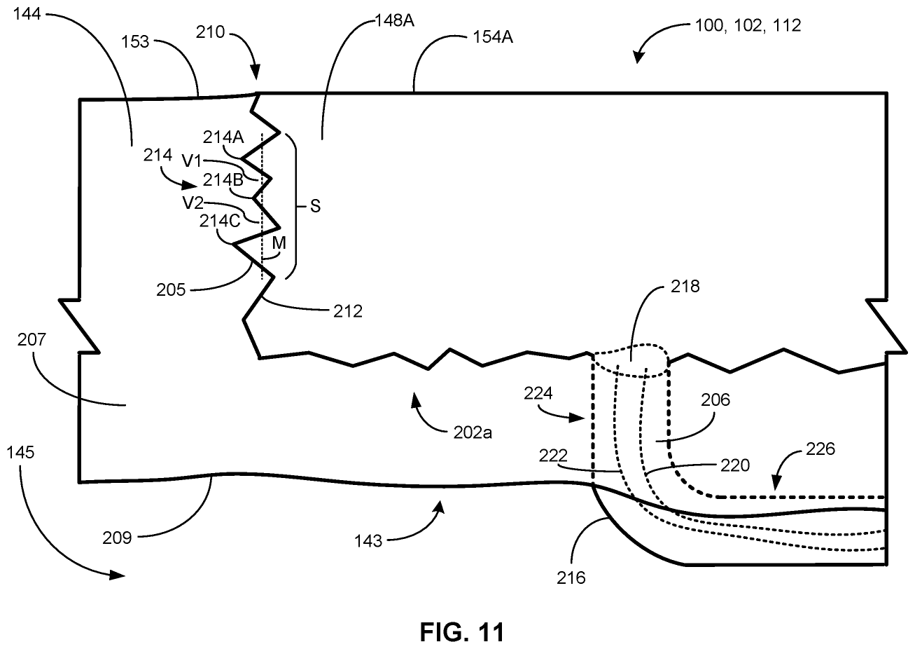

FIG. 11 schematically illustrates a cross-section of elongate structure 143 including a portion of material boundary 210 of FIG. 10. FIG. 11 illustrates the cross-section with a cutting plane passing through longitudinal axis 149 of FIG. 9. Material boundary 210 includes and/or is defined by jacket edge surface 205 of outer jacket 144 and electrode edge surface 212 of electrode 148A. Electrode 148A is seated within reduced diameter segment 202a. Electrode edge surface 212 has a surface roughness such that electrode edge surface 212 defines a plurality of asperities 214 (“asperities 214”), such as asperity 214a, asperity 214b, and asperity 214c. As used herein, surface roughness may mean that the asperities 214 cause a displacement of electrode edge surface 212 from a mean line M to vary over some length S. Electrode edge surface 212 may define, for example, an arithmetic average roughness value describing an average deviation of electrode edge surface 212 from mean line M, or may define some other surface roughness measure. In FIG. 10, asperities 214, electrode edge surface 212, and jacket edge surface 205 are not necessarily to scale and may substantially define a different profiles than those schematically illustrated at FIG. 10.

In examples, jacket edge surface 205 intrudes into one or more volumes (e.g., cavities) defined by asperities 214, such as volume V1 defined by asperity 214a and asperity 214b, volume V2 defined by asperity 214b and 214c, or some other volume defined by the asperities of electrode edge surface 212. Jacket edge surface 205 may intrude into volumes V1, V2, V3 to enable and/or increase a mechanical adhesion between jacket edge surface 205 and electrode edge surface 212. In examples, jacket edge surface 205 and electrode edge surface 212 are substantially mechanically interlocked. The mechanical adhesion (e.g., interfacial adhesion) thus enabled and/or increased may assist in retaining electrode 148A seated within reduced diameter segment 202a. The mechanical adhesion thus enabled and/or increased may limit motion of electrode 148A when electrode 148A is seated within reduced diameter segment 202a to, for example, assist in maintaining electrode 148A firmly seated when catheter 102 and/or elongate structure 143 is navigated within the vasculature of a patient, and/or when neuromodulation element 112 is in a low profile delivery state, a radially expanded deployed state, and/or transitioning between the low profile delivery state and the radially expanded deployed state.

In examples, outer jacket 144 comprises a polymer having one or more material properties causing the polymer to exhibit a fluid-like (or more fluid-like) state when a condition of the polymer (e.g., a temperature) is altered. For example, the polymer be a thermoplastic and may exhibit a more fluid-like behavior (e.g., a decrease in viscosity) as a temperature of the polymer increases. In examples, the polymer may substantially exhibit the properties of a solid (e.g., a viscoelastic and/or soft solid) which may have a very high viscosity at a first temperature and exhibit properties of a fluid having a lower viscosity at a second temperature (e.g., a softening temperature or glass transition temperature) greater than the first temperature. In examples, when in the fluid-like state, the polymer may wet electrode edge surface 212, such that the polymer substantially contacts and spreads over the substrate (e.g., asperities 214) defined by electrode edge surface 212. The wetting may cause jacket edge surface 205 to intrude into volumes V1, V2, V3 to enable and/or increase a mechanical adhesion between jacket edge surface 205 and electrode edge surface 212. In examples, material boundary 210 is formed by reflowing the polymer (e.g., by heating outer jacket 144 to the second temperature) such that the polymer wets electrode edge surface 212 in a fluid-like state. Subsequent to the wetting, the polymer may be allowed to reset to a more solid-like state (e.g., by cooling or allowing outer jacket 144 to cool to the first temperature). The polymer may densify (e.g., increase its specific density) when the polymer resets to the more solid-like state, such that electrode edge surface 212 substantially intrudes into volumes V1, V2, V3 in a substantially solid state.

In examples, elongate structure 143 mechanically supports electrodes 148A, 148B, 148C such that active areas 154A, 154B, 154C substantially protrude relative to some portion of jacket exterior surface 153. For example, outer jacket 144 may include one or more transition sections such as transition section 318 described for jacket exterior surface 308 and active area 154A (FIG. 13) and/or transition section 426 described for jacket exterior surface 408 and active area 154A, 154B, 154C (FIG. 16).

As discussed, neuromodulation element 112 may include one or more electrical leads electrically coupled to an electrode, such as electrical lead 216 electrically coupled to electrode 148A. Electrical lead 216 may extend at least partially through inner jacket lumen 145. In examples, slot 206 is configured to provide a passage for electrical lead 216 from inner jacket lumen 145 through jacket body 207 to enable the electrical connection between electrical lead 216 and electrode 148A. In examples, electrical lead 216 is electrically coupled to electrode 148A at a coupling point 218. Slot 206 may be configured to provide the passage for electrical lead 216 when electrode 148A is seated in reduced-diameter segment 202a. In examples, electrical lead 216 includes a wire 220 configured to transfer energy to electrode 148A. In examples, electrical lead 216 includes a second wire 222 defining a wire pair. Wire 220 and/or second wire 222 may be electrically coupled (e.g., welded, soldered, etc.) to electrode 148A to form coupling point 218. In examples in which the neuromodulation element 112 includes a wire pairs (e.g., wire 220 and second wire 222), coupling point 218 may be configured to function as a thermocouple for sensing (e.g., measuring) a temperature of and/or surrounding electrode 148A. These temperature measurements may be used to inform a required amount of power to be generated (e.g., by generator 104 of FIG. 1) during a neuromodulation procedure.

Outer jacket 144 may at least partially mechanically support electrical lead 216. In examples, catheter 102 may define a material boundary 224 adjoining electrical lead 216 and outer jacket 144 to assist in holding electrical lead 216 more securely in place to, for example, assist in maintaining the electrical connection provided by and/or physical integrity of coupling point 218. Material boundary 224 may enable a mechanical adhesion between outer jacket 144 and electrical lead 216. In examples, material boundary 224 includes and/or is defined by a portion of jacket edge surface 205 (e.g., a portion different than that defining material boundary 210) and an outer-most surface of electrical lead 216 (e.g., an outermost surface of an insulative cover of electrical lead 216). The portion of jacket edge surface 205 defining material boundary 224 may be, for example, defined by a portion of jacket body 207. Jacket edge surface 205 may be in contact with the outer-most surface of electrical lead 216 around at least some portion of a perimeter defined by electrical lead 216 when outer jacket 144 mechanically supports electrical lead 216 (e.g., as electrical lead 216 is electrically coupled to coupling point 218). In some examples, jacket edge surface 205 substantially envelops at least a portion of electrical lead 216 (e.g., the portion extending through slot 206).

In examples, jacket edge surface 205 intrudes into one or more volumes (e.g., cavities) defined by asperities of a surface of electrical lead 216 to enhance the mechanical adhesion between outer jacket 144 and electrical lead 216. Jacket edge surface 205 may intrude into volumes defined by the surface asperities of electrical lead 216 in substantially the same manner as that described for material boundary 210. For example, in the fluid-like state, the polymer comprising outer jacket 144 may wet the surface of electrical lead 216 such that the polymer substantially contacts and spreads over the substrate defined by the surface of electrical lead 216. The wetting may cause jacket edge surface 205 to intrude into volumes defined by the surface asperities of electrical lead 216 to enable and/or increase a mechanical adhesion between jacket edge surface 205 and electrical lead 216. Material boundary 224 may be formed by reflowing the polymer (e.g., by heating outer jacket 144 to the second temperature) to wet the surface of electrical lead 216, followed by allowing the polymer to reset to a more solid-like state (e.g., by cooling or allowing outer jacket 144 to cool to the first temperature). The mechanical adhesion (e.g., interfacial adhesion) thus enabled and/or increased may assist in maintaining the electrical connection provided by and/or physical integrity of coupling point 218 to, for example, assist in maintaining the electrical connection when catheter 102 and/or elongate structure 143 is navigated within the vasculature of a patient, and/or when neuromodulation element 112 is in a low profile delivery state, a radially expanded delivery state, and/or transitioning between the low profile delivery state and the radially expanded delivery state.

In some examples, slot 206 defines an initial area and/or initial cross-sectional dimension prior to the formation of material boundary 226 and a subsequent area and/or subsequent cross-sectional dimension polymer subsequent to the formation of material boundary 226. The initial area may be greater than the subsequent area and/or the initial cross-sectional dimension may be greater than the subsequent cross-sectional dimension. For example, slot 206 may define the initial area and/or initial cross-sectional dimension to add in the installation and/or threading through of electrical lead 216 through slot 206 during an assembly. Subsequent to installation and/or threading through, formation of material boundary 224 may cause slot 206 to define the subsequent area and/or subsequent cross-sectional area to, for example, increase the mechanical adhesion between outer jacket 144 and electrical lead 216 (e.g., when outer jacket 144 substantially envelops electrical lead 216). Slot 206 may define any initial area and/or initial cross-sectional dimension and may define any initial shape, including circular, ovalur, or another shape. In examples, slot 206 defines an initial shape configured to cause outer jacket 144 to substantially envelop electrical lead 216 when slot 206 defines the subsequent area and/or subsequent cross-sectional dimension (e.g., subsequent to a reflow of the polymer).

In some examples, catheter 102 may define a material boundary 226 adjoining electrical lead 216 and jacket interior surface 209 to assist in holding electrical lead 216 more securely in place. Material boundary 224 may enable a mechanical adhesion between outer jacket 144 and electrical lead 216. In examples, material boundary 224 includes and/or is defined by a portion of jacket interior surface 209 and the outer-most surface of electrical lead 216. Jacket interior surface 209 may be in contact with the outer-most surface of electrical lead 216 around at least some portion of a perimeter defined by electrical lead 216 (e.g., as electrical lead 216 is electrically coupled to coupling point 218). In some examples, jacket interior surface 209 substantially envelops at least a portion of electrical lead 216 (e.g., the portion extending through slot 206). In some examples, jacket interior surface 209 intrudes into one or more volumes defined by asperities of the surface of electrical lead 216 in same manner as that described for the intrusion of jacket edge surface 205 into the surface asperities of electrical lead 216. For example, material boundary 226 may be formed by reflowing the polymer comprising outer jacket 144, followed by allowing the polymer to reset to a more solid-like state. The mechanical adhesion (e.g., interfacial adhesion) thus enabled and/or increased may assist in maintaining the electrical connection provided by and/or physical integrity of coupling point 218 and/or mitigate and/or eliminate wrapping and/twisting of electrical lead 216 and other leads within neuromodulation element 112 when catheter 102 and/or elongate structure 143 is navigated within the vasculature of a patient, and/or when neuromodulation element 112 is in a low profile delivery state, a radially expanded delivery state, and/or transitioning between the low profile delivery state and the radially expanded delivery state.

Elongate structure 143 of FIG. 6, FIG. 10, and FIG. 11 may be formed by removing some portion of a substrate including a polymer to define outer jacket 144. The substrate may define an inner lumen. In examples, the substrate defines a tubular structure. One or more portions of the substrate may be removed (e.g., using laser ablation) to define one or more of reduced diameter segments 202. Additional material may be removed from the substrate to define one or more of slots 206, such that outer jacket 144 defines jacket exterior surface 153, jacket interior surface 209, jacket inner lumen 145, one or more of reduced diameter segments 202A, 202B, 202C, 202D, and one or more of slots 206. One of more of electrodes 148A may be threaded over and/or otherwise positioned on jacket exterior surface 153 to seated within one of reduced diameter segments 202A, 202B, 202C, 202D. Electrical lead 216 may be threaded through slot 206. Electrical lead 216 may be coupled to one of electrode 148 (e.g., electrode 148A, electrode 148B, electrode 148C, and/or electrode 148D) prior to or subsequent to seating the one of electrodes 148 within one of reduced diameter segments 202A, 202B, 202C, 202D. The outer jacket 144 including one or more of electrodes 148 seated within one or more of reduced diameter segments 202A, 202B, 202C, 202D may be treated (e.g., heat treated) to cause a polymer comprising outer jacket 144 to reflow, generating material boundary 210, material boundary 224, and/or material boundary 226. The polymer comprising outer jacket 144 may densify during and/or subsequent to the treatment. Elongate structure 143 may be formed in this manner to have any number of electrodes and any number of electrical leads.

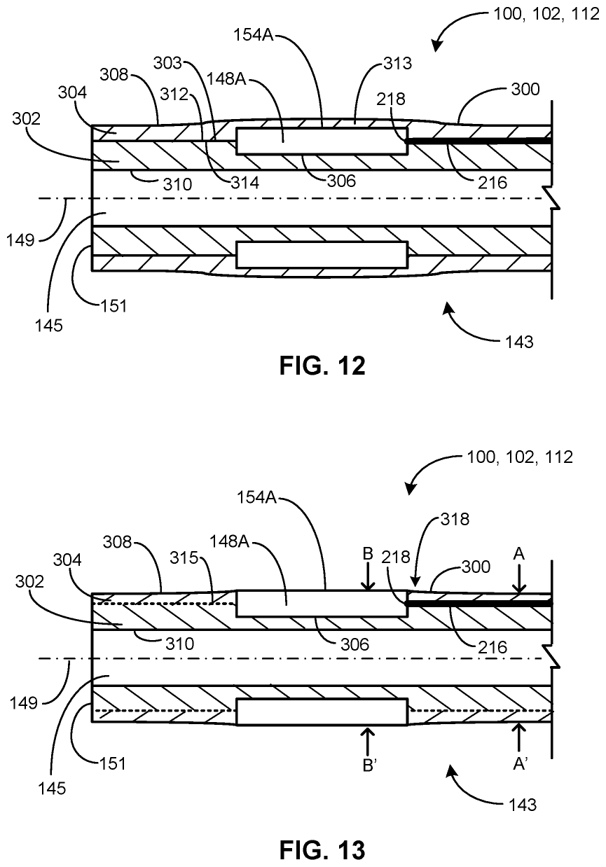

FIG. 12 is a cross-sectional view of an outer jacket 300 of a neuromodulation element 112 configured in accordance with some examples of the present disclosure. Outer jacket 300 includes an internal jacket member 302 defining a boundary 303 with an external jacket member 304. Electrode 148A is mechanically supported between internal jacket member 302 and external jacket member 304. External jacket member 304 may define jacket exterior surface 308. Internal jacket member 302 may define jacket interior surface 310. In the example of FIG. 12, electrical lead 216 is positioned between internal jacket member 302 and external jacket member 304, although this is not required. FIG. 13 is a cross-sectional view of outer jacket 300 with internal jacket member 302 defining a material boundary 305 with an external jacket member 304. Electrode 148A is seated in a reduced diameter segment 306 defined at least partially by internal jacket member 302 and external jacket member 304. Electrode 148A is mechanically supported within reduced diameter segment 306 such that active area 154A and jacket exterior surface 308 face outward from longitudinal axis 149. Outer jacket 300, jacket exterior surface 308, jacket interior surface 310, and reduced diameter segment 306 may be an examples of outer jacket 144, jacket exterior surface 153, jacket interior surface 209, and reduced diameter segment 202A respectively. Outer jacket 300 may be used in neuromodulation element 112 in place of outer jacket 144. Accordingly, outer jacket 300 may be described below in conjunction with components of catheter 102.

In the example of FIG. 12, external jacket member 304 may be substantially overlaid over internal jacket member 302 such that contact between a first surface 312 of external jacket member 304 and a second surface 314 of internal jacket member 302 provides an initial degree of mechanical adhesion between external jacket member 203 (e.g., first surface 312) and internal jacket member 302 (e.g., second surface 314). For example, frictional or other forces which arise mainly due to contact between first surface 312 and second surface 314 may provide the initial degree of mechanical adhesion. In examples, boundary 303 is an contact boundary defined by first surface 312 and second surface 314. In examples, external jacket member 304 comprises a first quantity of polymer and internal jacket member 302 includes a second quantity of polymer (the same or a different polymer from the first quantity), and boundary 303 extends between first surface 312 and second surface 314 to separate the first quantity of polymer and the second quantity of polymer. In examples, when external jacket member 304 is substantially overlaid over internal jacket member 302, external jacket member 304 may include a portion 313 (“exterior jacket portion 313”) covering some portion of or substantially all of active area 154A of electrode 148A.

FIG. 13 may be an example of FIG. 12 following a treatment (e.g., a heat treatment) of outer jacket 300. The treatment may substantially transform boundary 303 into a material boundary 315 between internal jacket member 302 and external jacket member 304. Material boundary 315 may enable a subsequent degree of mechanical adhesion between external jacket member 304 and internal jacket member 302 greater than the initial degree of mechanical adhesion. In some examples of material boundary 315, first surface 312 intrudes into one or more volumes defined by asperities of second surface 314 (or vice-versa) in same manner as that described for the intrusion of jacket edge surface 205 into the surface asperities of electrode edge surface 212. In some examples of material boundary 315, the first quantity of polymer comprising external jacket member 304 is intermingled with the second quantity of polymer comprising internal jacket member 302 includes a second quantity of polymer, such that material boundary 315 defines an interfacial boundary between external jacket member 304 and internal jacket member 302 comprising the first quantity of polymer and the second quantity of polymer.

In examples, material boundary 315 is formed by reflowing a polymer comprising external jacket member 304 and/or internal jacket member 302 (e.g., by heating outer jacket 300) such that the polymer wets first surface 213 and/or second surface 314, and/or such that the first quantity of polymer intermingles with the second quantity of polymer. Subsequent to the reflow, the polymer may be allowed to reset to a more solid-like state (e.g., by cooling) The polymer may densify (e.g., increase its specific density) when the polymer resets to the more solid-like state, such that first surface 312 intrudes into one or more volumes defined by asperities of second surface 314 (or vice-versa) in the substantially solid state, and/or such that the first quantity of polymer intermingles with the second quantity of polymer in the substantially solid state. In examples, portions of external jacket portion 313 remaining over active area 154A of electrode 148A may be removed, such that active area 154A may emit energy to a wall of an anatomical lumen without interference from external jacket portion 313. In examples, when external jacket member 304 and/or internal jacket member 302 reflow to form material boundary 315, external jacket member 304 and/or internal jacket member 302 may substantially envelop electrical lead 216 to, for example, assist in holding electrical lead 216 more securely in place, assist in maintaining an electrical connection of and/or physical integrity of coupling point 218, to mitigate and/or eliminate wrapping and/twisting of electrical lead 216 and other leads, and/or for other reasons.

In examples, elongate structure 143 may mechanically support electrode 148A such that active area 154A substantially protrudes relative to some portion of jacket exterior surface 153, 308. Elongate structure 143 may be configured to cause active area 154A to substantially protrude relative to jacket exterior surface 153, 308 to, for example, enhance a proximity and/or contact between active area 154A and a vessel of an anatomical lumen when elongate structure 143 is in the anatomical lumen and neuromodulation element 112 is in a radially expanded deployed state. For example, as illustrated in FIG. 13, jacket exterior surface 153, 308 may define a first cross-sectional dimension (e.g., between the arrows A-A′) and electrode 148A may define a second cross-sectional dimension (e.g., between the arrows B-B′) greater than the first cross-sectional dimension, such that that active area 154A substantially protrudes relative to a portion of jacket exterior surface 153, 308. In examples, the first cross-sectional dimension and/or the second cross-sectional dimension may be substantially perpendicular to longitudinal axis 149. In examples, the first cross-sectional dimension may be an outer diameter of outer jacket 144, 300, and/or the second cross-sectional dimension may be an outer diameter of electrode 148A.

In examples, outer jacket 144, 300 defines a transition section 318 substantially adjacent electrode 148A, with transition section 318 substantially tapering from the first cross-section dimension defined by jacket exterior surface 153, 308 to the second cross-sectional dimension defined by electrode 148A. In examples, transition section 318 defines a cross-sectional dimension substantially equal to the second cross-sectional dimension defined by electrode 148A. In some examples, transition section 318 includes some portion of material boundary 210 between outer jacket 144, 300 and electrode 148A. Transition section 318 may serve to cause active area 154A to substantially protrude relative to jacket exterior surface 153, 308 as material boundary 210 limit motion of electrode 148A when electrode 148A is seated within reduced diameter segment 202a, 306. In examples, the transition of transition section 318 from the first cross-sectional dimension to substantially cross-sectional dimension reduces, eliminates, and/or mitigates discontinuities (e.g., steps) between jacket exterior surface 153, 308 and active area 154A. The reduction, elimination, and/or mitigation may, for example, improve patient comfort as neuromodulation element 112 transits through and/or resides in an anatomical lumen of the patient.

Elongate structure 143 of FIG. 13 may be formed by loading internal jacket member 302 onto a mandrel or other supporting structure. Electrode 148A may be threaded over and/or otherwise positioned on second surface 314 of internal jacket member 302 (e.g., seated within at least some portion of reduced diameter segment 306). Electrical lead 216 may also be positioned on second surface 314 of internal jacket member 302 and, in examples, electrically connected to electrode 148A at coupling point 218. External jacket member 304 may subsequently be positioned over internal jacket member 302, electrode 148A, and/or electrical lead 216 such that electrode 148A and/or electrical lead 216 reside between internal jacket member 302 and external jacket member 304 to substantially define outer jacket 300 having the configuration of FIG. 12. The outer jacket 300 including electrode 148A may be treated (e.g., heat treated) to cause a polymer comprising external jacket member 304 and/or internal jacket member 302 to reflow, generating material boundary 315 and/or causing external jacket member 304 and/or internal jacket member 302 to substantially envelop electrical lead 216. The polymer comprising external jacket member 304 and/or internal jacket member 302 may densify during and/or subsequent to the treatment to define transition section 318. Portions of external jacket portion 313 remaining over active area 154A of electrode 148A may be removed (e.g., by laser ablation). An assembly comprising outer jacket 300, electrode 148A, and electrical lead 216 may be removed from the mandrel and/or other supporting structure. Elongate structure 143 may be formed in this manner to have any number of electrodes and any number of electrical leads.

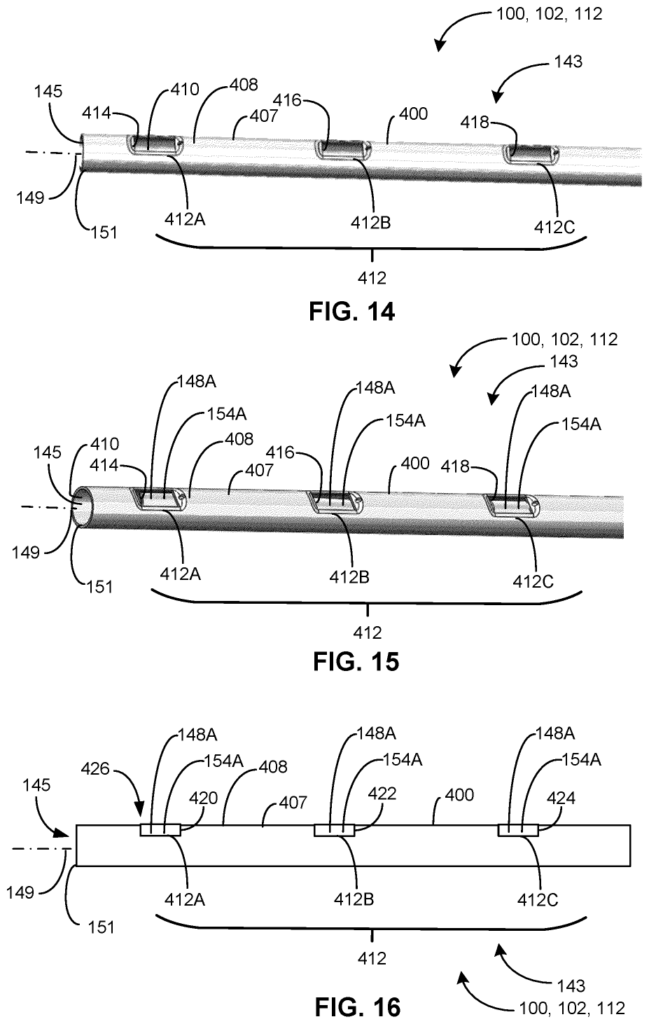

FIG. 14 is a perspective view of an outer jacket 400 of a neuromodulation element 112 configured in accordance with some examples of the present disclosure. Outer jacket 400 may include jacket body 407 defining jacket exterior surface 408 and jacket interior surface 410. Jacket interior surface 410 may define jacket inner lumen 145. Jacket body 407 may define one or more windows 412, such as window 412A, window 412B, and windows 413C. Each of windows 412 defines a passage from jacket inner lumen 145 to jacket exterior surface 408. Each of windows 412 may be defined by a boundary defining the passage. For example, window 412A may be defined by a window boundary 414 defining a first passage, window 412B may be defined by a window boundary 416 defining a second passage, and/or window 412C may be defined by a window boundary 418 defining a third passage. Jacket body may define any number of windows. Outer jacket 400, jacket body 407, jacket exterior surface 408, and jacket interior surface 410 may be examples of outer jacket 144, 300, jacket body 207, jacket exterior surface 153, 308, and jacket interior surface 209, 310 respectively.

Each of window 412A, window 412B, and/or window 412C may define an arc around jacket exterior surface 408 subtended by an angle having a vertex substantially located on longitudinal axis 149. Each of window boundary 414, window boundary 416, and/or window boundary 418 may define the arc. The angle may be any angle sufficient to define a window defining a passage from jacket inner lumen 145 to jacket exterior surface 408. In some examples, the angle is an angle from about 90 degrees to about 270 degrees. In some examples, the angle is an angle of about 180 degrees. Windows 412A, 412B, 412C may be located relative to each other in any arrangement. In examples, two or more of windows 412A, 412B, 412C are located such that two or more of active areas 148A, 148B, 148C face in a direction away from central axis 147 (FIGS. 3-4) when neuromodulation element 112 is in a radially expanded deployed state. In examples, two or more of windows 412A, 412B, 412C are located such that at least some portion of active area 154A, at least some portion of active area 154B, and/or at least some portion of active area 154C are substantially parallel to longitudinal axis 149 when neuromodulation element 112 is in the low-profile delivery state. In some examples, two or more of windows 412A, 412B, 412C are located such that a vector parallel to longitudinal axis 149 passes through the two or more of windows 412A, 412B, 412C when neuromodulation element 112 is in a low-profile delivery state and/or in a radially expanded deployed state.

FIG. 15 is a perspective view of outer jacket 400 with one or more electrodes such as electrode 148A, electrode 148B, and electrode 148C mechanically supported by elongate structure 143 (e.g., outer jacket 400). As before, electrode 148A defines active area 154A, electrode 148B defines active area 154B, and electrode 148C defines active area 154C. Electrodes 148A-148C are mechanically supported such that the passage defined by window 412A leaves active area 154A uncovered by jacket exterior surface 408, the passage defined by window 412B leaves active area 154B uncovered by jacket exterior surface 408, and/or the passage defined by window 412C leaves active area 154C uncovered by jacket exterior surface 408. Electrodes 148A-148C may be mechanically supported in any manner sufficient to leave active area 154A, 154B, 154C uncovered by jacket exterior surface 408. In examples, electrodes 148A-148C are mechanically supported by an internal jacket member within jacket inner lumen 145, such as internal jacket member 302 (FIGS. 12-13). One or more electrical leads (not shown) may be electrically connected to one or more of electrodes 148A, 148B, 148C. An electrical lead may extend through jacket inner lumen 145, between an internal jacket member such as internal jacket member 302 and an external jacket member such as external jacket member 304, and/or some other arrangement where the electrical lead may be electrically connected to at least one of electrodes 148A-148C.

FIG. 16 may be an example of FIG. 15 following a treatment (e.g., a heat treatment) of outer jacket 400. The treatment may substantially transform window boundary 414 into a material boundary 420 adjoining electrode 148A and outer jacket 400, substantially transform window boundary 416 into a material boundary 422 adjoining electrode 148B and outer jacket 400, and/or substantially transform window boundary 418 into a material boundary 424 adjoining electrode 148C and outer jacket 400. A jacket edge surface such as jacket edge surface 205 (FIGS. 10-11) may define each of material boundaries 420, 422, 424. Material boundaries 420, 422, 424 may have substantially similar structure to material boundary 210 defined by jacket edge surface 205 and electrode 148A (FIGS. 10-11). Hence, material boundary 420 may enhance a mechanical adhesion of electrode 148A and outer jacket 400, material boundary 422 may enhance a mechanical adhesion of electrode 148B and outer jacket 400, and/or material boundary 424 may enhance a mechanical adhesion of electrode 148C and outer jacket 400. In examples, elongate structure 143 mechanically supports electrodes 148A, 148B, 148C such that active areas 154A, 154B, 154C substantially protrude relative to some portion of jacket exterior surface 408. For example, outer jacket 400 may include one or more transition sections such as transition section 426. Transition section 426 may be configured relative to jacket exterior surface 408 and electrodes 148A, 148B, 148C in substantially the same manner as transition section 318 is configured relative to jacket exterior surface 308 (FIG. 13).

Elongate structure 143 of FIG. 16 may be formed by removing some portion of a substrate including a polymer to define outer jacket 400. The substrate may define an inner lumen. In examples, the substrate defines a tubular structure. One or more portions of the substrate may be removed (e.g., using laser ablation) to define one or more of windows 412A, 412B, 412C, such that outer jacket 400 defines jacket exterior surface 408, jacket interior surface 410, jacket inner lumen 145, and one or more of windows 412A, 412B, 412C. Additional material may be removed from the substrate to define one or more slots such as slots 206 (FIGS. 5-10). One of more of electrodes 148A may be positioned within jacket inner lumen 145 such that at least one of windows 412A, 412B, 412C leaves at least one of active area 154A, 154B, 154C uncovered by jacket exterior surface 408. In examples, one of more of electrodes 148A, 148B, 148C is threaded over and/or otherwise positioned on a second surface (e.g., second surface 314) of an internal jacket member (e.g., internal jacket member 302), and may be seated within at least some portion of a reduced diameter segment (e.g., reduced diameter segment 306) defined by the internal jacket member. Electrical lead 216 may be coupled to one of electrodes 148A, 148B, 148C, prior to or subsequent to positioning the one of electrodes 148A, 148B, 148C within jacket inner lumen 145. The outer jacket 400 including one or more of electrodes 148A, 148B, 148C may be treated (e.g., heat treated) to cause the polymer comprising outer jacket 144 to reflow, generating material boundary 420, material boundary 422, and/or material boundary 424. The polymer comprising outer jacket 400 may densify during and/or subsequent to the treatment. Elongate structure 143 may be formed in this manner to have any number of electrodes and any number of electrical leads.

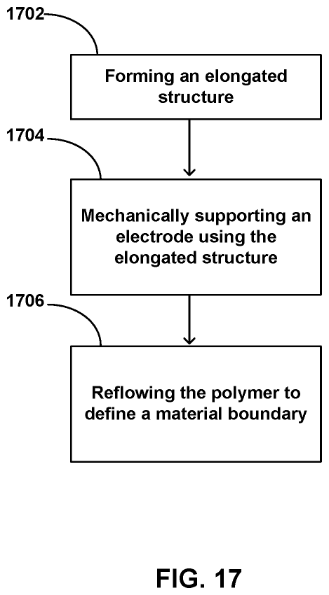

An example technique for forming a neuromodulation element 112 is illustrated in FIG. 17. Although the technique is described mainly with reference to medical system 100 of FIGS. 1-16, the technique may be applied to other medical systems in other examples.

The technique includes forming an elongate structure 143 of a neuromodulation element 112 defining a longitudinal axis 149 (1702). Elongate structure 143 may be configured to have a substantially linear shape when neuromodulation element 112 is in a low-profile delivery state and a coiled shape when neuromodulation element 112 is in a radially expanded deployed state. Elongate structure 143 may include an outer jacket 144, 300, 400 comprising a polymer. Outer jacket 144, 300, 400 may define a jacket exterior surface 153, 308, 408. Outer jacket 144, 300, 400 may include a jacket interior surface 209, 310, 410 defining a jacket inner lumen 145.

The technique includes mechanically supporting at least one of electrodes 148 using elongate structure 143 (1704). Electrode 148A, 148B, 148C, 148D may define an active surface 154A, 154B, 154C, 154D configured to emit energy. Elongate structure 143 may mechanically support electrode 148A, 148B, 148C, 148D such that jacket exterior surface 153, 308, 1408 and active area 154A, 154B, 154C, 154D face outward from longitudinal axis 149.

In examples, elongate structure 143 is formed by removing some portion of a substrate including a polymer to define outer jacket 144, 300, 400. In examples, portions of the substrate are removed using laser ablation. The substrate may define jacket inner lumen 145. In examples, the substrate defines a tubular structure. One or more portions of the substrate may be removed (e.g., using laser ablation) to define one or more of reduced diameter segment 202A, 202B, 202C, 202D, reduced diameter segment 306, one or more of windows 412A, 412B, 412C, and/or one or more slots 206. One of more of electrodes 148A, 148B, 148C, 148D may be threaded over and/or otherwise positioned on jacket exterior surface 153 to seat within one of reduced diameter segments 202A, 202B, 202C, 202D. One or more of electrodes 148A, 148B, 148C, 148D may be threaded over and/or otherwise positioned on second surface 314 of internal jacket member 302 (e.g., to seat within reduced diameter segment 306). In examples, electrode 148A, 148B, 148C, 148D is positioned between first surface 312 of external jacket member 304 and second surface 314 of internal jacket member 302. One of more of electrodes 148A, 148B, 148C, 148D may be positioned within jacket inner lumen 145 such that at least one of windows 412A, 412B, 412C leaves at least one of active area 154A, 154B, 154C uncovered by jacket exterior surface 408.

In examples, elongate structure 143 may be formed by initially loading the substrate or internal jacket member 302 onto a mandrel or other supporting structure. Electrical lead 216 may be threaded through slot 206 and/or positioned between first surface 312 and second surface 314. Electrical lead 216 may be electrically coupled to one of electrodes 148A, 148B, 148C, 148D prior to or subsequent to seating the one of electrodes 148A, 148B, 148C, 148D within one of reduced diameter segments 202A, 202B, 202C, 202D, 306 and/or positioning electrical lead 216 between first surface 312 and second surface 314. In examples, the technique includes threading electrical lead 216 through slot 206.

The technique may include reflowing the polymer to define a material boundary (1706). Reflowing the polymer may include defining material boundary 210, 224, 226, 305. Reflowing the polymer may include treating (e.g., heat treating) outer jacket 144, 300, 400 and electrode 148A, 148B, 148C, 148 D when elongate structure mechanically supports electrodes 148. Reflowing the polymer may cause outer jacket 144, 300, 400 to substantially envelop electrical lead 216. The technique may include setting the polymer (e.g., by cooling and/or allowing the polymer to cool). The polymer may densify during and/or subsequent to the treatment. In examples, the technique includes reflowing the polymer to define transition section 318,426. The technique may include removing portions of external jacket portion 313 remaining over active areas 154A, 154B, 154C, 154D (e.g., by laser ablation). 34. In examples, reflowing the polymer includes causing the polymer to wet a surface of electrode 148A, 148B, 148C, 148D, electrical lead 216, and/or external jacket member 304 and/or internal jacket member 302. Reflowing the polymer may cause the polymer to intrude into volumes defined by asperities of one or more of jacket edge surface 205, electrode edge surface 212, electrical lead 216, first surface 312, and/or second surface 314.

The technique may include inserting shape memory structure 142 into jacket inner lumen 145 defined by elongate structure 143.

Catheters configured in accordance with at least some embodiments of the present technology can be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include an electrode-based treatment modality alone or in combination with another treatment modality. Electrode-based treatment can include delivering electricity and/or another form of energy to tissue at or near a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity.

Heating effects of electrode-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° Celsius (C)) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

The following clauses are useful to understand the invention:
1. A catheter comprising a neuromodulation element, the neuromodulation element comprising:
   an elongate structure defining a longitudinal axis, wherein the elongate structure includes an outer jacket comprising a polymer, and wherein the outer jacket defines an exterior surface of the elongate structure; and
   at least one electrode configured to emit energy from an active area, wherein the elongate structure mechanically supports the at least one electrode such that the exterior surface and the active area face outward from the longitudinal axis, and wherein the catheter defines a material boundary adjoining the outer jacket and the at least one electrode, wherein the material boundary enables a mechanical adhesion between the polymer and the at least one electrode.
2. The catheter of clause 1, wherein the at least one electrode comprises one electrode of a plurality of electrodes, wherein the plurality of electrodes are spaced longitudinally apart from each other along the longitudinal axis of the elongate structure.
3. The catheter of clause 1 or clause 2, wherein the at least one electrode defines a plurality of asperities along the material boundary, and wherein the polymer intrudes into one or more volumes defined by the plurality of asperities to enable the mechanical adhesion.
4. The catheter of clause 3, wherein:
   the polymer defines a flow temperature;
   the polymer is configured to flow into the one or more volumes when the polymer is heated to the flow temperature; and
   the polymer is configured to remain in the one or more volumes when the polymer is cooled to a temperature less than the flow temperature.
5. The catheter of any one of clauses 1-4, wherein the polymer is mechanically interlocked with a surface of the at least one electrode at the material boundary.
6. The catheter of any one of clauses 1-5, wherein the material boundary adjoins the exterior surface of the outer jacket and the active area of the at least one electrode.
7. The catheter of any one of clause 1-6, wherein the elongate structure mechanically supports the at least one electrode such that the active area faces outward from a central axis defined by the neuromodulation element when the neuromodulation element is in a radially expanded deployed configuration.
8. The catheter of any one of clause 1-7, wherein the polymer defines a flow temperature, and wherein the polymer is configured to wet a surface of the at least one electrode at the material boundary when the polymer has a temperature at least equal to the flow temperature.
9. The catheter of any one of clauses 1-8, wherein the at least one electrode comprises a band electrode extending circumferentially around the longitudinal axis.
10. The catheter of any one of clauses 1-9, wherein the material boundary defines a circumference extending around the longitudinal axis.
11. The catheter of any one of clauses 1-10,
   wherein the elongate structure defines one or more reduced-diameter segments spaced longitudinally apart along the longitudinal axis of the elongate structure, and
   wherein the at least one electrode is seated in a reduced-diameter segment of the one or more reduced-diameter segments.
12. The catheter of clause 11, wherein the outer jacket includes an internal jacket member and an external jacket member contacting the internal jacket member, and wherein the internal jacket member and the external jacket member define at least one of the reduced-diameter segments.
13. The catheter of clause 11 or clause 12, wherein the reduced-diameter segment is fully circumferential.
14. The catheter of any one of clauses 1-13,
   wherein the outer jacket defines a jacket lumen and a window defining a passage from the jacket lumen to the exterior surface of the outer jacket,
   wherein the at least one electrode is positioned at least partially within the jacket lumen, and
   wherein the elongate structure mechanically supports the at least one electrode such that the window substantially surrounds the active area of the at least one electrode.
15. The catheter of clause 14, wherein the material boundary surrounds the window.
16. The catheter of any one of clauses 1-15,
   wherein the catheter is convertible between a low-profile delivery configuration and a radially expanded deployed configuration, and
   wherein the elongate structure is configured to have a substantially linear shape when the neuromodulation element is in the low-profile delivery configuration and configured to have a substantially helical shape when the neuromodulation element is in the radially expanded deployed configuration.
17. The catheter of clause 16, further comprising a shape memory structure pre-formed to urge the elongate structure toward the substantially helical shape.
18. The catheter of clause 17, wherein the shape memory structure includes a Nitinol coil.
19. The catheter of any one of clauses 1-18, further comprising an electrical lead electrically coupled to the at least one electrode,
   wherein the outer jacket defines an inner lumen, and
   wherein the electrical lead extends through the inner lumen.
20 The catheter of clause 19, further comprising a slot defining a passage from the inner lumen to the at least one electrode when the elongate structure mechanically supports the at least one electrode, wherein the electrical lead extends through the slot.
21. The catheter of clause 20, wherein the catheter defines a material boundary adjoining the outer jacket and the electrical lead when the electrical lead extends through the slot, wherein the material boundary adjoining the outer jacket and the electrical lead enables a mechanical adhesion between the polymer and the electrical lead.
22. The catheter of any one of clauses 19-22, wherein an interior surface of the outer jacket opposite the exterior surface defines the inner lumen, and wherein the catheter defines a wire material boundary adjoining the interior surface and the electrical lead when the electrical lead extends through the inner lumen, wherein the material boundary enables a mechanical adhesion between the polymer and the electrical lead.
23. The catheter of any one of clauses 19-22, wherein the electrical lead comprises a wire pair electrically coupled to the at least one electrode at a coupling point, and wherein the coupling point comprises a thermocouple.
24. The catheter of any one of clauses 19-23,
   wherein the at least one electrode is one of a plurality of electrodes,
   wherein the electrical lead is one of a plurality of electrical leads, and
   wherein each electrical lead is electrically coupled to at least one electrode.
25. The catheter of clause 24,
   wherein the neuromodulation element defines a central axis,
   wherein each individual electrode in the plurality of electrodes defines a corresponding active area, and
   wherein the elongate structure mechanically supports the each individual electrode to cause the corresponding active area to face away from the central axis when the neuromodulation element is in a radially expanded deployed configuration.
26. The catheter of clause 25, wherein the each individual electrode is spaced from a neighboring electrode in the plurality of electrodes by a spacing displacement defined along the longitudinal axis, and wherein the spacing displacement is defined to cause the individual active area to face away from the central axis when the neuromodulation element is in the radially expanded deployed configuration.
27. The catheter of any one of clauses 19-26, wherein the outer jacket defines a first cross-sectional dimension substantially perpendicular to the longitudinal axis and the at least one electrode defines a second cross-sectional dimension substantially perpendicular to the longitudinal axis, wherein the second cross-sectional dimension is greater than the first cross-sectional dimension.
28. The catheter of clause 27, wherein the outer jacket defines a transition section adjacent the at least one electrode, wherein the transition section defines a cross-sectional dimension at least equal to the second cross-sectional dimension.
29. A method of forming a neuromodulation element, comprising:
   forming an elongate structure defining a longitudinal axis, wherein the elongate structure includes an outer jacket comprising a polymer, and wherein the outer jacket defines an exterior surface of the elongate structure;
   mechanically supporting, using the elongate structure, at least one electrode configured to emit energy from an active area such that the exterior surface and the active area face outward from the longitudinal axis, and
   reflowing the polymer to define a material boundary adjoining the outer jacket and the at least one electrode, wherein the material boundary enables a mechanical adhesion between the polymer and the at least one electrode.
30. The method of clause 29, further comprising mechanically supporting, using the elongate structure, a plurality of electrodes, wherein the plurality of electrodes are spaced longitudinally apart from each other along the longitudinal axis of the elongate structure.
31. The method of clause 29 or clause 30, further comprising reflowing the polymer to cause the polymer to flow into one or more volumes defined by a plurality of asperities defined by the at least one electrode to enable the mechanical adhesion.
32 The method of clause 31, further comprising:
   heating the polymer to a flow temperature to reflow the polymer into the one or more volumes; and
   cooling the reflowed polymer to a temperature less than the flow temperature.
33. The method of clauses 29-32, wherein the material boundary adjoins the exterior surface of the outer jacket and the active area of the at least one electrode.
34. The method of any one of clauses 29-33, further comprising heating the polymer to decrease a polymer viscosity of the polymer.
35. The method of any one of clauses 29-34, further comprising causing the polymer to wet a surface of the at least one electrode at the material boundary.
36. The method of any one of clauses 29-35, wherein the at least one electrode comprises a band electrode, and further comprising surrounding the longitudinal axis circumferentially using the band electrode.
37. The method of any one of clauses 29-36, further comprising extending the material boundary circumferentially around the longitudinal axis.
38. The method of any one of clauses 29-37, further comprising seating the at least one electrode in a reduced-diameter segment of the elongate structure.
39. The method of catheter of clause 38, further comprising defining the reduced-diameter segment by adjoining an internal jacket member of the outer jacket and an external jacket member of the outer jacket.
40. The method of any one of clauses 29-39, further comprising mechanically supporting the at least one electrode such that a window defining a passage from a lumen of the outer jacket to the exterior surface of the outer jacket substantially surrounds the active area of the at least one electrode.
41. The method of clause 40, further comprising defining the material boundary around the window.
42. The method of any one of clauses 29-41, further comprising inserting a pre-formed shape memory structure into a lumen defined by the elongate structure to urge the elongate structure toward the helical state.
43. The method of any one of clauses 29-42, further comprising:
   electrically coupling an electrical lead to the at least one electrode, and
   extending the electrical lead through an inner lumen defined by the outer jacket.
44. The method of clause 43, further comprising extending the electrical lead through a slot defining a passage from the inner lumen to the at least one electrode.
45. The method of clause 44, further comprising reflowing the polymer to define a material boundary adjoining the outer jacket and the electrical lead.
46. The method of any one of clauses 43-45, further comprising reflowing the polymer to define a wire material boundary adjoining the electrical lead and an interior surface of the outer jacket defining the inner lumen.
47. The method of any one of clauses 43-46, further comprising defining a thermocouple by electrically coupling a wire pair of the electrical lead to the at least one electrode at a coupling point.
48. The method of any one of clauses 43-47, wherein the at least one electrode is one of a plurality of electrodes, and wherein the electrical lead is one of a plurality of electrical leads, and further comprising electrically coupling each electrical lead to at least one electrode.
49. The method of clause 48, wherein each individual electrode in the plurality of electrodes defines an individual active area, and further comprising mechanically supporting, using the elongate structure, the each individual electrode to cause the individual active area to face away from a helix axis when the neuromodulation element is in a radially expanded deployed configuration.
50. The method of clause 51, wherein the each individual electrode is spaced from a neighboring electrode in the plurality of electrodes by a spacing displacement defined along the longitudinal axis, and further comprising defining the spacing displacement to cause the individual active area to face away from the helix axis when the neuromodulation element is in the radially expanded deployed configuration.
51. The method of any one of clauses 29-50,
   wherein the catheter is convertible between a low-profile delivery configuration and a radially expanded deployed configuration, and
   wherein the elongated structure is configured to have a substantially linear shape when the neuromodulation element is in the low-profile delivery configuration and configured to have a helical shape when the neuromodulation element is in the radially expanded deployed configuration.
52. The method of clause 51, further comprising positioning a shape memory structure relative to the elongate structure, wherein the shape memory structure is configured to urge the elongate structure from the low-profile delivery configuration to the radially expanded deployed configuration when the shape memory structure is positioned relative to the elongated structure.

1. A catheter comprising a neuromodulation element, the neuromodulation element comprising:
an elongate structure defining a longitudinal axis, wherein the elongate structure includes an outer jacket comprising a polymer, and wherein the outer jacket defines an exterior surface of the elongate structure; and
at least one electrode configured to emit energy from an active area,
wherein the elongate structure mechanically supports the at least one electrode such that the exterior surface and the active area face outward from the longitudinal axis, and wherein the catheter defines a material boundary adjoining the outer jacket and the at least one electrode, wherein the material boundary enables a mechanical adhesion between the polymer and the at least one electrode.

2. The catheter of claim 1, wherein the at least one electrode defines a plurality of asperities along the material boundary, and wherein the polymer intrudes into one or more volumes defined by the plurality of asperities to enable the mechanical adhesion.

3. The catheter of claim 2, wherein:
the polymer defines a flow temperature;
the polymer is configured to flow into the one or more volumes when the polymer is heated to the flow temperature; and
the polymer is configured to remain in the one or more volumes when the polymer is cooled to a temperature less than the flow temperature.

4. The catheter of claim 1, wherein the polymer is mechanically interlocked with a surface of the at least one electrode at the material boundary.

5. The catheter of claim 1, wherein the elongate structure mechanically supports the at least one electrode such that the active area faces outward from a central axis defined by the neuromodulation element when the neuromodulation element is in a radially expanded deployed configuration.

6. The catheter of claim 1, wherein the at least one electrode comprises a band electrode extending circumferentially around the longitudinal axis.

7. The catheter of claim 1,
wherein the elongate structure defines one or more reduced-diameter segments spaced longitudinally apart along the longitudinal axis of the elongate structure, and
wherein the at least one electrode is seated in a reduced-diameter segment of the one or more reduced-diameter segments.

8. The catheter of claim 1,
wherein the outer jacket defines a jacket lumen and a window defining a passage from the jacket lumen to the exterior surface of the outer jacket,
wherein the at least one electrode is positioned at least partially within the jacket lumen, and
wherein the elongate structure mechanically supports the at least one electrode such that the window substantially surrounds the active area of the at least one electrode.

9. The catheter of claim 1,
wherein the neuromodulation element is convertible between a low-profile delivery configuration and a radially expanded deployed configuration, and
wherein the elongate structure is configured to have a substantially linear shape when the neuromodulation element is in the low-profile delivery configuration and configured to have a substantially helical shape when the neuromodulation element is in the radially expanded deployed configuration.

10. The catheter of claim 1, further comprising:
an electrical lead electrically coupled to the at least one electrode; and
a slot defining a passage from an inner lumen of the outer jacket to the at least one electrode when the elongate structure mechanically supports the at least one electrode,
wherein the electrical lead extends through the inner lumen, and
wherein the electrical lead extends through the slot.

11. The catheter of claim 10, wherein the electrical lead comprises a wire pair electrically coupled to the at least one electrode at a coupling point, and wherein the coupling point comprises a thermocouple.

12. The catheter of claim 1, wherein the outer jacket defines a first cross-sectional dimension substantially perpendicular to the longitudinal axis and the at least one electrode defines a second cross-sectional dimension substantially perpendicular to the longitudinal axis, wherein the second cross-sectional dimension is greater than the first cross-sectional dimension.

13. The catheter of claim 12, wherein the outer jacket defines a transition section adjacent the at least one electrode, wherein the transition section defines a cross-sectional dimension at least equal to the second cross-sectional dimension.

14. A method, comprising:
forming an elongate structure defining a longitudinal axis, wherein the elongate structure includes an outer jacket comprising a polymer, and wherein the outer jacket defines an exterior surface of the elongate structure;
mechanically supporting, using the elongate structure, at least one electrode configured to emit energy from an active area such that the exterior surface and the active area face outward from the longitudinal axis, and
reflowing the polymer to define a material boundary adjoining the outer jacket and the at least one electrode, wherein the material boundary enables a mechanical adhesion between the polymer and the at least one electrode.

15. The method of claim 14, wherein reflowing the polymer comprises heating the polymer to a flow temperature to cause the polymer to reflow into one or more volumes defined by a plurality of asperities defined by the at least one electrode to enable the mechanical adhesion, the method further comprising cooling the reflowed polymer to a temperature less than the flow temperature.

16. The method of claim 14, further comprising causing the polymer to wet a surface of the at least one electrode at the material boundary.

17. The method of claim 14, further comprising seating the at least one electrode in a reduced-diameter segment of the elongate structure.

18. The method of catheter of claim 17, further comprising defining the reduced-diameter segment by adjoining an internal jacket member of the outer jacket and an external jacket member of the outer jacket.

19. The method of claim 14, further comprising mechanically supporting the at least one electrode such that a window defining a passage from an inner lumen of the outer jacket to the exterior surface of the outer jacket substantially surrounds the active area of the at least one electrode.

20. The method of claim 14, further comprising:
extending an electrical lead through a slot defining a passage from an inner lumen defined by the outer jacket to the at least one electrode;
electrically coupling the electrical lead to the at least one electrode, and extending the electrical lead through the inner lumen.

21. A catheter comprising:
an elongate structure defining a longitudinal axis, wherein the elongate structure includes an outer jacket comprising a polymer, wherein the outer jacket defines an exterior surface of the elongate structure, wherein the elongate structure defines a plurality of reduced-diameter segments spaced longitudinally apart along the longitudinal axis, and wherein the outer jacket defines an inner lumen;
a plurality of electrodes each configured to emit energy from a respective active area,
wherein each electrode of the plurality of electrodes is seated in a respective reduced-diameter segment of the plurality of reduced-diameter segments, and
wherein the elongate structure mechanically supports the plurality of electrodes such that the exterior surface and the active areas face outward from the longitudinal axis, and wherein the catheter defines a plurality of material boundaries adjoining the outer jacket and the plurality of electrodes, wherein the plurality of material boundaries enable a mechanical adhesion between the polymer and the plurality of electrodes; and
a plurality of electrical leads, each electrical lead of the plurality electrically coupled to a respective electrode of the plurality of electrodes, wherein the plurality of electrical leads extend through the inner lumen.

22. The catheter of claim 21, wherein at least one electrical lead of the plurality of electrical leads comprises a wire pair electrically coupled to a respective electrode at a coupling point, and wherein the coupling point comprises a thermocouple.

23. The catheter of claim 21,
wherein the elongate structure is convertible between a low-profile delivery configuration and a radially expanded deployed configuration,
wherein the elongate structure is configured to have a substantially linear shape when the elongate structure is in the low-profile delivery configuration and configured to have a substantially helical shape when the elongate structure is in the radially expanded deployed configuration, and
wherein the elongate structure mechanically supports the plurality of electrodes such that the active areas face outward from a central axis defined by the elongate structure when the elongate structure is in the radially expanded deployed configuration.
